(19) United States
(12) Patent Application Publication
(10) Pub. No.: US 2026/0273053 A1
(43) Pub. Date: Sep. 17, 2026

(54) HIGHLY CONCENTRATED ANTIBODY COMPOSITIONS

(71) Applicant: SANOFI, Paris (FR)

(72) Inventors: Vasco FILIPE, Paris (FR); Yatin R. GOKARN, Cambridge, MA (US); Xin JIN, Cambridge, MA (US); Mostafa NAKACH, Paris (FR); Sanket PATKE, Cambridge, MA (US); Atul SALUJA, Cambridge, MA (US); Bertrand WOINET, Paris (FR)

(21) Appl. No.: 18/873,990

(22) PCT Filed: Jun. 15, 2023
(86) PCT No.: PCT/EP2023/066133

§ 371 Date: Dec. 11, 2024

Related U.S. Application Data
(60) Provisional application No. 63/352,438, filed on Jun. 15, 2022.

(30) Foreign Application Priority Data
Jul. 11, 2022 (EP) 22315141.6

(51) Int. Cl. A61K 39/395; A61K 47/18; A61K 47/26

(52) U.S. Cl. CPC A61K 39/39591; A61K 47/183; A61K 47/26

(57) ABSTRACT

Provided are methods for producing an aqueous antibody composition comprising at least 200 mg/ml antibody as well as methods for producing a dried antibody composition. Further provided are high concentration antibody compositions. Further provided is the use of the provided antibody compositions in medicine.

### TECHNICAL FIELD OF THE INVENTION

Provided are methods for producing an aqueous antibody composition comprising at least 200 mg/ml antibody as well as methods for producing adried antibody composition. Further provided are high concentration antibody compositions. Further provided is the use of the provided antibody compositions in medicine.

### BACKGROUND

Antibodies are multifunctional components of the immune system. In the past decades, a large number of monoclonal antibodies have been approved as medicinal products.

Monoclonal antibodies (or fragments thereof) are directed against different antigens were generated and are used in medicine, for example for the treatment of immunologic diseases and of cancer.

The marginal stability of antibodies, in particular of highly concentrated antibodies, combined with the desire to have long-term stability has been a driver for developing methods capable of stabilizing proteins. Dehydration of the protein formulation resulting in dry powders and addition of excipients are the two most common stabilizing techniques to prolong shelf-life. Protein stability can be significantly enhanced by removing water from the formulation. This is a result of reduced mobility of the protein and to the absence of certain degradation pathways facilitated by water. Under the right excipients and drying conditions, proteins are incorporated in an amorphous matrix, and this amorphous matrix is maintained as long as the temperature is maintained below the glass transition temperature, thereby enhancing the long-term stability of the protein. In addition, cryoprotectants such as trehalose and sucrose stabilize the protein by excluding water from the vicinity of the protein and provide protection against degradation during drying, upon storage of dried powder, and on storage of the aqueous solution upon rehydration. To circumvent denaturation at the air-water interface surfactants are often used to prevent protein adsorption at the interface.

There is a need for antibody formulations having a good long-term stability and appropriate powder characteristics. The long-term stability of the dried powder is primarily dependent on the water content (typically less 5%), formulation, the protein structure, and the storage conditions. While drying is often necessary to achieve a long-term stable product, the process of drying can introduce a combination of thermal, interfacial, and mechanical stresses that may negatively impact protein stability. Optimized drying process parameters and addition of excipients are required to reduce the impact of these stresses on the product. Addition of excipients alters the effect of the drying process and makes the process protein and formulation specific.

Several techniques can be used to dry proteins and obtain protein powders. This includes drying technologies such as freeze-drying or lyophilization, spray drying, spray freeze drying or prilling, and super critical fluid drying. All these technologies use different physical principles for desiccation, induces different stresses during drying, and also result in powders with different particle or powder properties.

Gikanga et al. disclose methods for manufacturing of high-concentration monoclonal antibody formulations via spray drying (Gikanga et al., PDA J Pharm Sci Technol. 2015 January-February; 69(1):59-73).

Deokar et al. provide comparison of different techniques to achieve high concentration (~200 mg/mL) antibody formulations (Deokar et al. J Pharm Sci. 2020; 109(12):3579-3589).

Clénet D, Hourquet V, Woinet B, Ponceblanc H, Vangelisti M. A spray freeze dried micropellet based formulation proof-of-concept for a yellow fever vaccine candidate. Eur J Pharm Biopharm. 2019 September; 142:334-343.

WO 2018/204374 A1 discloses aqueous antibody formulations.

US 2020/390705 A1 discloses spray dried antibody formulations.

U.S. Pat. No. 11,351,256 B2, WO 2013/016648 A2 and WO 2013/063510 A1 disclose methods to obtain an antibody formulation.

However, many problems can occur with pharmaceutical antibody formulations at high concentrations, e.g. stability challenges such formation of visible and sub-visible particles and antibody aggregates and solution property challenges such as high turbidity. It is important that the antibody formulations for parenteral use have low aggregate levels (up to 5% high molecular weight species or HMWS acceptable based on commonly accepted industry specifications), low sub-visible particles (meets USP <787> specifications, Allowable levels: 10 μm particles ≤6000 particles per container, 25 μm particles ≤600 particles per container), low turbidity (up to 40 NTU based on marketed product benchmarking, Kingsbury J S et al. J Pharm Sci. 2021 September; 110(9):3176-3182. doi: 10.1016/j.xphs.2021.05.005. Epub 2021 May 15. PMID: 34004217), and no visible particles. USP <787> is a standard established by the United States Pharmacopeial Convention (USP), USP chapter <787>, Subvisible Particulate Matter in Therapeutic Protein Injections.

Therefore, it is important to avoid the formation of antibody aggregates (which can be measured by measuring the change of the percentage of high molecular weight species). While it is important that the final antibody formulation that the end user utilizes has stability attributes that meet specifications, the stability of the antibody during drying, during storage of the dried intermediate product (e.g. a powder or a micropellets) at recommended storage temperature and shelf-life, and during storage as a solution at recommended storage temperature and shelf-life must be ensured in order to have a commercially viable product.

There is a need for highly concentrated antibody pharmaceutical formulations, e.g. for formulations comprising at least 200 mg/ml antibody, in particular if large doses of antibodies are to be administered, for example by subcutaneous administration via use of pre-filled syringes. Ability to achieve formulations comprising of 200 mg/mL or more antibody will allow higher amount of antibody drug substance in a single pre-filled syringe and so administration of higher doses via subcutaneous administration instead of intravenous administration or reduce the number of subcutaneous injections in some cases, both of which will enhance the patient-centricity of the therapy.

Thus, there remains a need for improved high concentration pharmaceutical antibody compositions which avoid these limitations and which meets typical acceptability specifications.

### SUMMARY OF THE PRESENT INVENTION

Provided herein is a method for producing an aqueous antibody composition I comprising at least 200 mg/ml antibody, said method comprising the steps a), b), and c)
a) providing an aqueous composition A comprising
   a1) an antibody in a concentration which is lower than the concentration of the antibody in the antibody composition I,
   a2) at least one surfactant,
   a3) at least one cryoprotectant,
   a4) at least 0.4% (w/v) arginine, or a salt thereof, wherein the weight
   (w) is calculated as arginine, based on the total volume of the aqueous composition A, such as 0.4 to 3.5% (w/v) arginine, wherein the ratio of the amount of a1) to the sum of the amounts of a3) and a4) is from 11:1 to 11:8, and, optionally
   a5) a buffer
b) drying the aqueous composition A provided in step a) to yield an antibody composition B, and
c) reconstituting the composition dried in step b) to give the aqueous antibody composition I.

In an embodiment of the provided method, the salt of arginine is selected from the group consisting of arginine-CI, arginine-aspartate, arginine-glutamate, arginine-sulfate, arginine-acetate, arginine-succinate and mixtures thereof.

In an embodiment of the provided method, the arginine is arginine-Cl.

In an embodiment of the provided method, the at least one cryoprotectant is selected from the group consisting of trehalose, sucrose, sorbitol, glycerol and mannitol.

In an embodiment of the provided method, the cryoprotectant is trehalose.

In an embodiment of the provided method, the ratio of the amount of a1) to the sum of the amounts of a3) and a4) is from 11:1 to 11:4, such as from 11:2 to 11:4.

In an embodiment of the provided method, the aqueous composition A comprises less than 200 mg/ml antibody, such as 10 to 190 mg/ml antibody, such as 20 to 170 mg/ml antibody, such as 50 to 150 mg/ml antibody, such as 80 to 120 mg/ml antibody.

In an embodiment of the provided method, the aqueous composition A comprises 150 mg/ml antibody.

In an embodiment of the provided method, the aqueous antibody composition I comprises more than 200 mg/ml antibody.

In an embodiment of the provided method, the aqueous antibody composition I comprises at least 250 mg/ml antibody, such as more than 250 mg/ml antibody.

In an embodiment of the provided method, the aqueous antibody composition I comprises at least 300 mg/ml antibody, such as such as more than 300 mg/ml antibody.

In an embodiment of the provided method, the aqueous antibody composition I comprises 230 mg/ml to 420 mg/ml antibody, such as 260 mg/ml to 400 mg/ml antibody.

In an embodiment of the provided method, the aqueous antibody composition I comprises 270 to 350 mg/ml antibody, such as 280 to 320 mg/ml antibody, such as 300 mg/ml antibody.

In an embodiment of the provided method, the antibody is a monoclonal antibody.

In an embodiment of the provided method, the at least one surfactant is a polysorbate, such as polysorbate 20 or 80, or a polaxamer, such as polaxamer 188.

In an embodiment of the provided method, the buffer is a histidine, citrate, acetate, phosphate, Tris, succinate or glycine buffer.

In an embodiment of the provided method, the pH of the composition A is about 5.0 to 8.0, such as about 5.5 to 7.

In an embodiment of the provided method, the antibody composition B is in the form of a powder or of micropellets.

In an embodiment of the provided method, the step b) is carried out by freeze drying or spray drying to give a freeze-dried or spray dried powder.

In an embodiment of the provided method, the step b) is carried out by spray freeze drying to give spray freeze-dried micropellets.

In an embodiment of the provided method, the freeze-dried powder is reconstituted in a reconstitution solution for a time period of 1.5 to 4.0 hours.

In an embodiment of the provided method, the spray dried powder is reconstituted in a reconstitution solution for a time period of at least four hours.

In an embodiment of the provided method, the spray freeze-dried micropellets are reconstituted in a reconstitution solution in a period of 1 to 2 hours.

In some embodiments, the powder or the micropellets are reconstituted in water. Thus, the reconstitution solution may be water.

In an embodiment of the provided method, the aqueous antibody composition I is a clear and transparent solution.

Further provided is an aqueous antibody composition I obtained or obtainable by the method for producing an aqueous antibody composition provided herein.

Further provided is an aqueous antibody composition I comprising at least 200 mg/ml antibody.

In an embodiment of the provided aqueous antibody composition I comprises 270 to 350 mg/ml antibody, such as 280 to 320 mg/ml antibody, such as 300 mg/ml antibody.

In another embodiment of the provided aqueous antibody composition I comprises 230 mg/ml to 420 mg/ml antibody, such as 260 mg/ml to 400 mg/ml antibody.

In an embodiment, the aqueous antibody composition I is a clear and transparent solution.

In an embodiment, the aqueous antibody composition I comprises at least 200 mg/ml antibody and has an opalescence below 40 NTU, such as an opalescence of below 25 NTU or below 15 NTU.

In an embodiment, the aqueous antibody composition I additionally comprises at least one surfactant, at least one cryoprotectant, such as trehalose, and arginine, or a salt thereof.

Further provided herein is a container comprising the aqueous antibody composition I provided herein.

In an embodiment of the provided container, the container is a prefilled syringe, a vial, cartridge, an ampule or an autoinjector. For example, the container may be an autoinjector or a prefilled syringe.

Also provided herein is a kit, comprising the container provided herein, and a label or instructions for the administration and use of the aqueous antibody composition I.

Further provided is an aqueous antibody composition I provided herein for use in medicine.

Also provided is a method of producing a dried antibody composition B,
a) providing an aqueous composition A comprising
   a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
   a2) at least one surfactant,
   a3) at least one cryoprotectant, and
   a4) at least 0.4% (w/v) arginine, or a salt thereof, wherein the weight
   (w) is calculated as arginine, based on the total volume of the aqueous composition A, such as 0.4 to 3.5% (w/v) arginine, wherein the ratio of the amount of a1) to the sum of the amounts of a3) and
   a4) is from 11:1 to 11:8, and
b) spray freeze drying the composition provided in step a) to give the dried antibody composition B.

In an embodiment, of the above method, the dried antibody composition B is in the form of micropellets.

Also provided is a dried antibody composition B, obtained or obtainable by the method of producing a dried antibody composition B and a container comprising said dried antibody composition B.

Also provided is a dried antibody composition B, and a container comprising said dried antibody composition B, the dried antibody composition B being in the form of micropellets, the composition B comprising
a1) an antibody,
a2) at least one surfactant,
a3) at least one cryoprotectant, such as trehalose, and
a4) arginine, or a salt thereof,
wherein the ratio of the amount of a1) to the sum of the amounts of a3) and a4) is from 11:1 to 11:8, such as from 11:2 to 11:8, wherein the weight of a4 is calculated as the free amino acid.

Also provided is a dried antibody composition B, and a container comprising said dried antibody composition B, the dried antibody composition B being in the form of a powder, the composition B comprising
a1) an antibody,
a2) at least one surfactant,
a3) at least one cryoprotectant, such as trehalose, and
a4) arginine, or a salt thereof,
wherein the ratio of the amount of a1) to the sum of the amounts of a3) and a4) is from 11:1 to 11:8, such as from 11:2 to 11:8, wherein the weight of a4 is calculated as the free amino acid.

Further provided is the use of the dried antibody composition B provided herein for producing an aqueous antibody composition I as defined herein comprising at least 200 mg/ml antibody.

Further provided is the use of the aqueous composition A for producing an aqueous antibody composition I as defined herein comprising at least 200 mg/ml antibody.

Further provided is an aqueous composition A as defined in connection with the method for producing an aqueous antibody composition I. The definitions apply accordingly.

Thus, provided is an aqueous composition A comprising:
a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
a2) at least one surfactant,
a3) at least one cryoprotectant, and
a4) at least 0.4% (w/v) arginine, or a salt thereof, wherein the weight (w) is calculated as arginine, based on the total volume of the aqueous composition A, such as 0.4 to 3.5% (w/v) arginine, wherein the ratio of the amount of a1) to the sum of the amounts of a3) and a4) is from 11:1 to 11:8.

### DESCRIPTION OF THE FIGURES

FIG. 1 Reconstituted solutions of eight different freeze-dried monoclonal antibodies at concentrations of 100, 150, 200, 250, 300, 350, and 400 mg/mL. Clear and transparent solution were obtained for all eight antibodies up to 300 mg/mL.

FIG. 2 Percentage of high molecular weight species (a) and opalescence (b) of reconstituted solutions from 100-350 mg/mL for eight monoclonal antibodies. Marginal changes in high molecular species were observed with increase in monoclonal antibody concentration with all values below 5%. Decrease in opalescence was observed with increase in monoclonal antibody concentration with all values <30 NTU.

FIG. 3 Increase of percentage of high molecular weight species during freeze drying. % HMWS were measured before and after freeze drying. Marginal increase in % HMWS were observed as a result of freeze-drying indicating protective role of excipients. Compositions containing at least 0.5% arginine-CI generally had lower increase in HMWS relative to trehalose only compositions.

FIG. 4 Increase of percentage of high molecular weight species of freeze-dried powder after storage at 40° C. Compositions containing at least 0.5% arginine-CI generally had lower increase in HMWS relative to trehalose only compositions. While 40° C. is not the intended storage condition, it is generally accepted as a condition for accelerated stability testing.

FIG. 5 Reconstituted solutions with 300 mg/mL monoclonal antibody A, B, D and E. An overview on the formulation can be found in Table 2. Clear and transparent aqueous solution with target protein concentration of 300 mg/mL were achieved for all shown antibodies and compositions.

FIG. 6 Change in % HMWS between antibody composition I (300 mg/mL) and antibody composition A (100 mg/mL) for monoclonal antibody A, B, D and E. Similar to observations presented in FIG. 2, marginal changes in high molecular species were observed with increase in monoclonal antibody concentration.

FIG. 7 Change in % HMWS between antibody composition I (300 mg/mL) after storage at 40° C. for 2 weeks. Marginal changes in high molecular species were observed upon short-term storage at 40° C. While 40° C. is not the intended storage condition, it is generally accepted as a condition for accelerated stability testing.

FIG. 8 Reconstituted solutions with 300 mg/mL monoclonal antibody A, B, D, E, G and H after using a variety of aqueous solutions for reconstitution. An overview on the formulations can be found in Example 3. Clear and transparent aqueous solution with target protein concentration of 300 mg/mL were achieved for all shown antibodies and compositions indicating suitability of a variety of aqueous solutions for reconstitution.

FIG. 9 Change in % HMWS after storage of antibody composition I (300 mg/mL) for two weeks at 40° C. An overview on the formulations can be found in Example 3. Reconstitution solutions containing glycine, arginine-CI, arginine-glutamate, magnesium-glutamate, combination of arginine-CI and glycine, and combination of arginine-CI and magnesium-glutamate are effective in providing protection. Marginal changes in high molecular species were observed upon short-term storage at 40° C. 40° C. is not the intended storage condition, it is generally accepted as a condition for accelerated stability testing.

FIG. 10 Spray freeze-dried micropellets and spray dried power for different formulations of monoclonal antibodies I and B. An overview on the formulations can be found in Example 4.

FIG. 11 Composition I (300 mg/mL) after reconstituting spray freeze-dried micropellets and spray dried power for different formulations of monoclonal antibodies I and B. Clear and transparent aqueous solution with target antibody concentration of 300 mg/mL were achieved for both antibodies following reconstitution of spray freeze-dried micropellets with opalescence <5 NTU for antibody B. While 300 mg/ml antibody solutions were achieved for both antibodies following reconstitution of spray dried powder, resulting solutions with cloudy with opalescence >30 NTU.

FIG. 12 Evaluation of short and long-term storage stability at different temperatures of the spray dried powder and spray freeze-dried micropellets. Marginal changes in high molecular species were observed with all values below 5%, indicating optimal storage stability.

FIG. 13 Evaluation of short storage and accelerated stability of the 300 mg/ml composition I resulting from reconstitution of spray dried powder and spray freeze-dried at different temperatures and for different storage periods. Marginal changes in high molecular species were observed at 5 and 25° C. with all values below 5%, indicating optimal storage stability. High starting values for antibody B may be attributed to analytical variability and sample handling.

### DEFINITIONS

Unless defined otherwise, all technical and scientific terms used herein have the same meaning as is commonly understood by one of ordinary skill in the art. It is noted here that as used in this specification and the appended claims, the singular forms “a,” “an,” and “the” also include plural reference, unless the context clearly dictates otherwise. Further, it will be understood that the term “at least one” as used herein means at one or more than one. For example, at two, three, four, or more. Depending on the item, the term refers to, the skilled person understands as to what upper limit the term may refer, if any.

The term “about” as used herein means that with respect to any number recited after said term an interval accuracy exists within which a technical effect can be achieved. Accordingly, “about” as referred to herein, for example, refers to the precise numerical value or a range around said precise numerical value of +20%, +15%, +10%, or +5%. In an embodiment, the term refers to the exact value.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

The aqueous composition A provided in step a) of the method of the present invention shall comprise an antibody.

The term “antibody” as used herein refers to immunoglobulins or immunoglobulin-like molecules. It includes by way of example and without limitation, IgA, IgD, IgE, IgG and

IgM, combinations thereof, and similar molecules produced during an immune response in any vertebrate, for example, in mammals such as goats, rabbits and mice, as well as non-mammalian species, such as shark immunoglobulins. The term includes synthetic antibodies, monoclonal antibodies, recombinantly produced antibodies, multispecific antibodies (including bi-specific antibodies), human antibodies, humanized antibodies, single-domain antibody (also referred to as immunoglobulin single variable domain (ISVD), e.g. Nanobody® molecule), including monospecific, bispecific, multispecific single-domain antibodies, chimeric antibodies, intrabodies, single-chain Fvs (scFv) (e.g., including monospecific, bispecific, etc.), camelized antibodies, Fab fragments, F(ab′) 2 fragments, disulfide-linked Fvs (sdFv), anti-idiotypic (anti-Id) antibodies, and epitope-binding fragments of any of the above. In particular, antibodies include immunoglobulin molecules and immunologically active portions of immunoglobulin molecules, i.e., antigen binding domains or molecules that contain an antigen-binding site that specifically binds to the antigen (e.g., six complementarity determining regions (CDRs) of an antibody). The antibodies can be of any class (e.g., IgG, IgE, IgM, IgD, IgA, and IgY), any subclass (e.g., IgG1, IgG2, IgG3, IgG4, IgA1, and IgA2), or any subclass (e.g., IgG2a and IgG2b) of immunoglobulin molecule. In some embodiments, the antibody is humanized. In certain embodiments, the antibody is an IgG antibody, such as an IgG1 or IgG4 antibody. In an embodiment, the antibody is an IgG4 antibody.

In an embodiment, the antibody, or fragment thereof, has been produced recombinantly.

The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. The term “Complementarity Determining Regions” (abbreviated CDRs) refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L (for Light Chain Complementary Determining Regions) or CDRL1, CDRL2, CDRL3 and CDR1-H, CDR2-H, CDR3-H (for Heavy Chain Complementary Determining Regions) or CDRH1, CDRH2, CDRH3, respectively. A conventional antibody antigen-binding site, therefore, includes six CDRs, comprising the CDR set from each of a heavy and a light chain variable region.

In an embodiment, the antibody is a monoclonal antibody. As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells.

In an embodiment, the antigen binding protein is a diabody. A diabody is a bivalent antibody which comprises two polypeptide chains. Each polypeptide chain comprises the variable heavy domains and variable light domains joined by a linker. In an embodiment, the two polypeptide chains of a diabody are identical. In an alternative embodiment, the two polypeptide chains have different amino acid sequences, provided that the two chains bind the same antigen (either the same epitope or different epitopes within the same antigen).

The term “antibody” may also encompass mixtures of antibodies, such as a mixture of two antibodies or more.

In an embodiment, the antibody is a therapeutic antibody, i.e. an antibody in use in therapy. For example, the antibody is selected from the group consisting of adalimumab, belimumab, atezolizumab, elotuzumab, infliximab, evolocumab, nivolumab, cetuximab, pembrolizumab, durvalumab, siltuximab, eculizumab, fremanezumab-vfrm, ofatumumab, omalizumab, galcanezumab-gnlm, rituximab, panitumumab, daratumumab, palivizumab, denosumab, mepolizumab, golimumab, trastuzumab, obinutuzumab, avelumab, ocrelizumab, pertuzumab, vedolizumab, ramucirumab, tocilizumab, secukinumab, olaratumab, necitumumab, guselkumab, ustekinumab, bevacizumab, alemtuzumab, certolizumab (pegol) and benralizumab.

In addition to antibody, the “composition” or “formulation” shall contain further ingredients in, optionally, the amounts as specified herein. Excipients refer to inert substances that are commonly used as a diluent, vehicle, preservative, binder, stabilizing agent, etc. for drugs and includes, but is not limited to, amino acids (e.g., arginine), fatty acids and phospholipids, surfactants (e.g., polysorbate, nonionic surfactant, etc.), saccharides (e.g., sucrose, maltose, trehalose, etc.). By “pharmaceutically acceptable excipient” is meant any inert substance that is combined with the antibody as referred to herein for preparing an agreeable or convenient dosage form. The “pharmaceutically acceptable excipient” is an excipient that is non-toxic to recipients at the dosages and concentrations employed and is compatible with other ingredients of the formulation including the monoclonal antibody.

In addition to a1) the antibody, the composition A further comprises, a2) at least one surfactant, a3) at least one cryoprotectant, a4) arginine, or a salt thereof. Moreover, the composition may comprise a5) a buffer. Typically, the composition comprises the excipients in an amount as specified elsewhere herein.

Surfactants are chemical compounds that interact and stabilize biological molecules and/or general pharmaceutical excipients in a formulation. Surfactants generally protect the binding agent from air/solution interface induced stresses and solution/surface induced stresses, which may otherwise result in protein aggregation. Surfactants may include, but are not limited to, polysorbates, glycerin, dicarboxylic acids, oxalic acid, succinic acid, fumaric acids, phthalic acids, and combinations thereof. Those skilled in the art are aware that other surfactants, e.g. non-ionic or ionic detergents, can be used as long as they are pharmaceutically acceptable, i.e., suitable for administration to subjects. The surfactant is, in some embodiments, a polysorbate. Examples of polysorbates include polysorbate 20, polysorbate 40, polysorbate 60, polysorbate 65, and polysorbate 80. In an embodiment, the polysorbate is polysorbate 80. In an embodiment, the polysorbate is polysorbate 20. Polysorbate 80, Polysorbate 20, and polaxamer 188 are the most commonly used surfactants in marketed parenteral products.

In some embodiments, the surfactant is a polaxamer, such as polaxamer 188.

Typically, the surfactant is present in composition A in an amount from about 0.001% to about 0.2% (w/v), based on the total volume of composition A. For example, the surfactant may be present in composition A in an amount of about 0.005% (w/v), about 0.006% (w/v), about 0.007% (w/v), about 0.008% (w/v), about 0.009% (w/v), about 0.01% (w/V), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v) and about 0.2% (w/v). In some embodiments, the surfactant is present in formulation A from about 0.01% to about 0.1% (w/v), about 0.02% to about 0.08% (w/v), or about 0.03% to about 0.07% (w/v), e.g. about 0.05% (w/v). For example, polysorbate 80 may be present in an amount of about 0.05% (w/v).

As the surfactant is present in composition A, it will be also present in composition I.

Cryoprotectants are chemical compounds that protect the antibody comprised by the composition from freezing damage, i.e. from damage that is caused by ice formation or due to adsorption to ice-liquid interface. Thus, a cryoprotectant provides stability to an antibody in a formulation to militate against freezing-induced stresses. A cryoprotectant may be a saccharide, such as trehalose, sucrose, glucose, mannitol, mannose, and lactose or a polymers, such as or polyethylene glycol. Typically, the cryoprotectant is selected from the group consisting of cryoprotectants consisting of trehalose, sucrose, sorbitol, glycerol and mannitol.

In some embodiments, the cryoprotectant is trehalose.

The formulations or aqueous composition as referred to herein shall comprise the amino acid arginine, or a salt thereof. Typically, the composition comprises a salt of arginine. In an embodiment, the arginine salt is selected from the group consisting of arginine-CI (also referred to as arginine-HCl), arginine-aspartate, arginine-glutamate, arginine-sulfate, arginine-acetate, arginine-succinate and mixtures thereof.

In an embodiment, the salt is arginine-Cl.

The aqueous composition may comprise a5) a buffer. A buffer is an agent that maintains a physiologically suitable pH. In addition, a buffer may enhance isotonicity and chemical stability of the formulation. For example, the buffer may be selected from a histidine, a citrate, an acetate, a phosphate, a Tris, a succinate, or a glycine buffer.

In some embodiments, the buffer is present in composition A in a concentration from about 0.5 mM to about 50 mM, e.g., about 5 mM to about 15 mM. For example, it may be present in a concentration of about 15 mM.

In certain embodiments, the composition A provided in step a) of the method provided herein may have a pH which ranges from about 5.0 to 8.0, such as from about 5.5 to about 7.0. For example, the pH of composition A may be about 5.5, about 5.7, about 5.9, about 6.1 about 6.1, about 6.3, about 6.5, about 6.7, about 6.9, and about 7.0. In some embodiments, the pH of composition A may range from about 5.8 to about 6.3. The pH of composition A may be measured by any means known to those of skill in the art. A means for measuring pH is using a pH meter. The pH of composition A may be adjusted using any means known in the art. Chemicals for altering the pH of composition A are hydrochloric acid (HCl) and sodium hydroxide (NaOH).

In some embodiments, the pH of the composition A is antibody-specific, i.e. chosen to provide optimal stability. For example, the pH may be below or above the isoelectric point (pi) of the antibody. The isoelectric point is the pH at which a particular molecule or surface carries no net electrical charge. In an embodiment, the pH-value of composition A is not at the isoelectric point of the antibody. For example, the pH-value of composition A may be at least 0.5 above or below the isoelectric point of the antibody.

In step b) of the method of the present invention, the composition A as provided in step a) shall be dried to yield an antibody composition B.

The term “drying” or “dehydrating” as used herein refers to the removal of water from the aqueous composition A, thereby generating composition B. Typically, the water content (and thus the moisture content) of the composition B is less than 10%, such as less than 5%, such as less than 3%. In an embodiment, the water content is between 0.5% and 5%, such as between 0.5% and 3%. The water content or moisture content is the quantity of water contained in composition B, typically measured by Karl-Fischer moisture analysis. Lower moisture content can be achieved by careful selection of drying process parameters. Lower moisture content will ensure better stability during storage of composition B. In the studies underlying the present invention, Karl Fischer was used to determine the residual water content. The oven temperature was set to 120° C. and air flow was set to 80 mL/min, the drift needed to be below 20 μg of water/minute to start the measurement. The stop criterion was to have a relative drift near 15 μg of water/minute. Hydranal coulomat AG-oven (Honeywell Fluka) reagent was used as the anolyte solution in the titration cell. Prior to testing samples, a 1% water standard (MilliporeSigma) was run to ensure the instrument was performing correctly. In an embodiment, the moisture content as referred to herein is measured by this method.

Drying can be achieved by any method deemed appropriate, for example, it can be achieved by technologies such as freeze-drying (or lyophilization), spray drying, and spray freeze drying. Typically, drying is achieved by freeze-drying, spray drying or spray freeze drying.

In an embodiment, the drying step is carried out by freeze-drying (frequently also referred to as “lyophilization”).

As used herein, the term “freeze-drying” (or “lyophilization” or “cryodesiccation”) refers to a method by the composition A is cooled to temperature at which the water in the composition frozen. Afterwards, the frozen water is removed by one or two drying steps, a primary drying step (which involves removal of unbound water by sublimation) and, optionally a subsequent secondary drying step (which involves removal of bound solvent by desorption).

In another embodiment, the drying step is carried out by spray drying.

Spray drying is a three-step process involving an atomization step followed by removal of the aqueous solvent by evaporation and subsequent powder collection. The protein feed solution is sprayed directly into a drying chamber, where the droplets come in contact with a hot drying gas such as air or nitrogen. The drying time is very short and can vary between a few seconds to minutes based on the scale of the spray dryer. After drying the powder is collected either by a cyclone or by a filter. Process parameters such as feed flow rate, drying air flow rate, atomization flow rate, and inlet temperature and formulation parameters such as excipients and solid concentration impact the water content, particle size, particle morphology, density, and protein stability. Mechanical stress during atomization, thermal stress during drying, and interfacial stress during overall processing can adversely impact product stability. An increase in drying temperature will decrease the moisture content, which will aid protein stability, but will at the same time introduce more thermal stress and potential denaturation.

In another embodiment, the drying step is carried out by spray-freeze drying.

Spray freeze drying (frequently also referred to as “prilling”) is a four-step process involving an atomization step, spray freezing of the droplets thereby “locking” in the spherical droplet shape, followed by freeze drying and subsequent collection of micropellets (see e.g. WO 2013/050156 A1, or WO 2009/109550 A1). While the physical and morphological properties of the obtained powder are mainly affected by the first two steps, the third step relates to the drying time and the total energy required to dry the product. Process parameters such as feed flow rate, nozzle configuration, freezing rate, freezing temperature, drying air flow rate, and pressure and formulation parameters such as excipients and solid concentration impact the water content, particle size, particle morphology, density, and protein stability. Mechanical stress during atomization, ice-related stresses during freezing, thermal stress during drying, and interfacial stress during overall processing can adversely impact product stability. This technique allows a high degree of control over the residual moisture content, mass density, and particle size. An example for spray-freeze drying is disclosed in the Examples section.

The composition B yielded in step b) may be a powder (i.e. a dried powder) or in the form of micropellets. For example, the micropellets may be regular spherical micropellets or particles having a diameter from about 400 μm to about 700 μm. The diameter may be measured by scanning electron microscopy (SEM).

The form of the composition B may depend on the drying method. In an embodiment, step b) is carried out by freeze drying or spray drying to give a (freeze-dried or spray dried) powder. In another embodiment, step b) is carried out by spray freeze drying to give spray freeze-dried micropellets.

In an embodiment, the composition B is reconstituted to give the aqueous antibody composition I.

In step c) of the method of the present invention, the dried composition B is reconstituted to give the aqueous antibody composition I, i.e., a composition comprising at least 200 mg/ml antibody.

Step c) can be carried out immediately after composition B has been obtained. In another embodiment, composition B is stored for a period of time prior to carrying out step c).

The reconstitution of composition B can be done in any aqueous (reconstitution) solution (e.g., an aqueous buffer) deemed appropriate.

In an embodiment, the reconstitution solution is an aqueous solution comprising at least one additive selected from the group consisting of water, glycine, arginine-CI, guanidinium-CI, arginine-glutamate and magnesium-glutamate.

The aqueous antibody composition I or composition B as referred to herein shall be a “stable” composition. A stable composition is a composition in which the antibody, therein essentially retains its physical stability, identity, integrity, and/or chemical stability, identity, integrity, and/or biological activity upon storage. Various analytical techniques for measuring protein stability are available in the art. Stability can be measured at a selected temperature and other storage conditions for a selected time period. The stability may be determined by characterizing aggregation or level of high molecular weight species using size exclusion chromatography. For example, an antibody “retains its physical stability” in a composition, if it shows no signs of significant increase in aggregation levels, precipitation, and/or denaturation upon visual examination of color and/or clarity and characterization of level of high molecular weight species. In some embodiments, when using the composition of the invention, 5% or less, typically 4% or less, typically 3% or less, more typically 2% or less, and particularly 1% or less of the antibodies forms aggregates, as measured by Size Exclusion Chromatography (e.g. as described in the Examples section) or any other suitable method for measuring aggregation formation. For example, an antibody is considered stable in a particular composition, if the antibody monomer has a purity of about 90% or more, typically about 95% or more or about 98% or more after a certain predetermined period of time under certain storage conditions in a particular formulation. In an embodiment, less than 5% of the antibodies form aggregates.

Chemical stability can be assessed by detecting and quantifying chemically altered forms of the protein. Chemical alteration may involve size modification (e.g., clipping), which can be evaluated using (HP) SEC, SDS-PAGE, and/or matrix-assisted laser desorption ionization/time-of-flight mass spectrometry (MALDI/TOF MS), for example. Other types of chemical alteration include charge alteration which can be evaluated by ion-exchange chromatography, for example.

The term “opalescence” (frequently also referred to as “turbidity”) refers to a solutions optical characteristic caused by scattering of light which can be explained by different scattering events such as Rayleigh or Mie Scattering. The opalescence of a solution can be, e.g., assessed as described in Kingsbury J S et al., J Pharm Sci. 2021 September; 110(9):3176-3182. doi: 10.1016/j.xphs.2021.05.005. Epub 2021 May 15. PMID: 34004217 (which herewith is incorporated by reference in its entirety). In an embodiment, composition A and aqueous antibody composition I as referred to herein have an opalescence below 40 NTU (Nephelometric Turbidity Units (NTU), such as an opalescence of below 25 NTU. For example, the opalescence may be below 15 NTU. In an embodiment, dried antibody composition A as referred to herein have an opalescence below 40 NTU (Nephelometric Turbidity Units (NTU), such as an opalescence of below 25 NTU. For example, the opalescence may be below 15 NTU. In an embodiment, aqueous antibody composition I as referred to herein have an opalescence below 40 NTU

(Nephelometric Turbidity Units (NTU), such as an opalescence of below 25 NTU. For example, the opalescence may be below 15 NTU.

The aqueous antibody composition I or the composition B as referred may be used in therapy. Accordingly, the composition may be administered to a subject who is in need thereof, i.e. a subject who suffers from a disease or disorder or who is at risk of suffering from a disease or condition.

If composition B is used in therapy, it is required to reconstitute the composition to yield the aqueous antibody composition I as described elsewhere herein.

In one embodiment, the term “disease or disorder” refers to any pathological or unhealthy state which can be treated by administering the formulation or composition provided herein.

The “subject” or “patient” may be a vertebrate. The term “subject” includes both humans and other animals, particularly mammals, and other organisms. Accordingly, herein the subject may be an animal such as a mouse, rat, hamster, rabbit, guinea pig, ferret, cat, dog, chicken, sheep, bovine species, horse, camel, or primate. In some embodiments, the subject is a mammal. In some embodiments, the subject is a primate. In some embodiments, the subject is human.

The terms “administer” or “administration” refers to the act of injecting or otherwise physically delivering a substance as it exists outside the body (e.g., a formulation of the invention) into a patient, such as by mucosal, intradermal, intravenous, subcutaneous, intramuscular delivery and/or any other method of physical delivery described herein or known in the art. In an embodiment, a composition is administered subcutaneously. In another embodiment, a composition is administered intravenously.

The compositions described herein are usually administered in therapeutically effective amounts. The term “therapeutically effective amount” is understood by the skilled person. In some embodiments, the term refers to an amount which achieves a desired therapeutic reaction or a desired therapeutic effect alone or together with further doses, optionally without causing or only minimally causing unacceptable or unwanted side-effects.

The term “treating” or “treatment”, as used herein, refers to the administration of a compound or composition or a combination of compounds or compositions to a subject in order to: prevent, ameliorate, or eliminate a disease and/or disorder as referred to herein, such as obesity, in a subject. Thus, the term encompasses both the treatment of an existing disease or disorder as referred to herein, or prevention of disease or disorder, i.e. prophylaxis. It will therefore be recognized that treatment as referred to herein may, in some embodiments, be prophylactic. In some embodiments, the term refers to the treatment of an existing disease or disorder as referred to herein. Thus, the subject is suffering from said disease or disorder.

### DETAILED DESCRIPTION OF THE PRESENT INVENTION

Provided herein is a method for producing an aqueous antibody composition I comprising at least 200 mg/ml antibody, said method comprising the steps a), b) and c),
a) providing an aqueous composition A comprising
   a1) an antibody in a concentration which is lower than the concentration of the antibody in the antibody composition I,
   a2) at least one surfactant,
   a3) at least one cryoprotectant,
   a4) at least 0.4% (w/v) arginine, or a salt thereof, wherein the weight
   (w) is calculated as arginine, such as 0.4 to 3.5% (w/v) arginine wherein the ratio of the amount of a1) to the sum of the amounts of a3) and a4) is from 11:1 to 11:8, and
b) drying the composition provided in step a) to yield an antibody composition B, and
c) reconstituting the composition dried in step b)
to give the aqueous antibody composition I.

The aqueous antibody composition I produced by the method of the present invention shall comprise a high concentration of an antibody, such as of a monoclonal antibody, typically a therapeutic monoclonal antibody.

Typically, the aqueous antibody composition I comprises at least 200 mg/ml antibody, for example more than 200 mg/ml antibody.

Also typically, the aqueous antibody composition I comprises at least 250 mg/ml antibody, for example at least 300 mg/ml antibody.

In an embodiment, the aqueous antibody composition I comprises 230 mg/ml to 420 mg/ml antibody, such as 260 mg/ml to 400 mg/ml antibody.

In an embodiment, the aqueous antibody composition I comprises 270 to 350 mg/ml antibody, such as 280 to 320 mg/ml antibody.

In an embodiment, the aqueous antibody composition I comprises 300 mg/ml antibody.

Typically, the aqueous antibody composition I is a clear and transparent solution. This can be assessed visually (by the appearance of the composition).

The provided method for producing an aqueous antibody composition I shall comprises the steps of
a) providing an aqueous composition A
b) drying the composition provided in step a) to yield an antibody composition B, and
c) reconstituting the composition dried in step b) to give the aqueous antibody composition I.

The aqueous composition provided in step a) shall comprise:
a1) an antibody in a concentration which is lower than the concentration of the antibody in the antibody composition I,
a2) at least one surfactant,
a3) at least one cryoprotectant,
a4) at least 0.4% (w/v) arginine, or a salt thereof, wherein the weight (w) is calculated as arginine, such as 0.4 to 3.5% (w/v) arginine
wherein the ratio of the amount of a1) to the sum of the amounts of a3) and a4) is from 11:1 to 11:8, and optionally a5) a buffer.

The concentration of the antibody in a1) shall be lower that than the concentration of the antibody in the aqueous antibody composition I. For example, if the concentration of the antibody in composition I is at least 200 mg/ml, the concentration of said antibody in composition A is lower than 200 mg/ml. In some embodiments, the concentration of the antibody in composition A is at least 5%, such as at least 10%, such as at least 20%, such as at least 20% lower than in composition I. For example, the concentration of the antibody in composition A is between 10 to 70%, such as between 30% to 60% lower than the concentration in composition I.

In an embodiment, the aqueous composition A comprises less than 200 mg/ml antibody.

In an embodiment, the aqueous composition A comprises 10 to 190 mg/ml antibody.

In an embodiment, the aqueous composition A comprises 20 to 170 mg/ml antibody.

In an embodiment, the aqueous composition A comprises 50 to 150 mg/ml antibody.

In an embodiment, the aqueous composition A comprises 80 to 120 mg/ml antibody.

In an embodiment, the aqueous composition A comprises 100 mg/ml antibody.

The term “surfactant” has been defined above. Moreover, typical concentrations of the surfactant are given above. In an embodiment, the at least surfactant is a polysorbate, such as polysorbate 80. In another embodiment, the surfactant is a polaxamer, such as polaxamer 188.

The term “cryoprotectant” has been defined above. For example, the cryoprotectant is selected from the group of cryoprotectants consisting of trehalose, sucrose, sorbitol, glycerol and mannitol. In an embodiment, the cryoprotectant is trehalose.

The aqueous composition A may comprise a5) a buffer as described above.

In an embodiment, the buffer is a histidine buffer.

In an alternative embodiment, the buffer is a citrate buffer.

In an alternative embodiment, the buffer is an acetate buffer.

In an alternative embodiment, the buffer is a phosphate buffer.

In an alternative embodiment, the buffer is a Tris buffer.

In an alternative embodiment, the buffer is a succinate buffer.

In an alternative embodiment, the buffer is a glycine buffer.

Moreover, in an embodiment, the pH value of the composition is about 5.0 to 8.0, such as about 5.5 to 7 (but might depend on the antibody).

Typically, the aqueous composition A comprises a4) at least 0.4% (w/v) arginine, or a salt thereof, calculated as arginine based on the total volume of composition A. For example, the aqueous composition A comprises 0.4 to 3.5% (w/v) arginine (as free base). In an embodiment, the aqueous composition A comprises 1.0 to 3.0% (w/w) arginine (as free base), such as 1.25 o 2.8% (w/w) arginine).

It is to be understood that in case, the aqueous composition A comprises an arginine salt, amount is also calculated as the amount of arginine free base present. Arginine free base has a molecular weight of 174 g/mol. The term “amount” typically refers to the weight.

In an embodiment, the ratio of the amount of a1) to the sum of the amounts of a3) and a4) is from 11:1 to 11:8. For example, the ratio may be about 11:1, about 11:2, about 11:3, about 11:4, about 11:5, about 11:6, about 11:7, about 11:8. The ratio of the amount of a1), i.e. the amount of the amount of the antibody, to the sum of the amounts of a3) and a4), i.e. of the excipients, is to be understood as a weight/weight (w/w) ratio.

In an embodiment, the ratio of the amount of a1) to the sum of the amounts of a3) and a4) is from 11:1 to 11:4.

In an embodiment, the ratio of the amount of a1) to the sum of the amounts of a3) and a4) is from 11:2 to 11:4.

In an embodiment, the ratio of the amount of a1) to the sum of the amounts of a3) and a4) is from 11:2 to 11:3.

In an embodiment, the ratio of the amount of a1) to the sum of the amounts of a3) and a4) is from 11:1 to 11:4.

In some embodiments, a4) is a salt of arginine. In one embodiment, it was shown that the present of at least 0.4% arginine in composition A enhances the stability of antibody during the drying process (See Examples).

In an embodiment, the arginine salt is arginine-CI (arginine-HCl, see also Examples section).

In an alternative embodiment, the arginine salt is arginine-aspartate.

In an alternative embodiment, the arginine salt is arginine-glutamate.

In an alternative embodiment, the arginine salt is arginine-sulfate.

In an alternative embodiment, the arginine salt is arginine-acetate.

In an alternative embodiment, the arginine salt is arginine-succinate.

In step b) of the method of the present invention, the aqueous composition A to yield an antibody composition B. After drying, the water content of the dried composition, i.e., composition B, is less than 10%, such as less than 5%, such as less than 3%.

In an embodiment, composition A is dried by freeze drying. Freeze drying typically yields a powder. Thus, composition B is provided in the form of a powder.

In an alternative embodiment, composition A is dried by spray drying. Spray drying typically yields a powder. Thus, composition B is provided in the form of a powder.

In an alternative embodiment, composition A is dried by spray-freeze drying. Spray freeze drying typically yields a powder. Thus, composition B is provided in the form of micropellets.

In step c) of the method of the present invention, the dried composition B is reconstituted to give the aqueous antibody composition I, i.e. a composition comprising at least 200 mg/ml antibody.

Step c) can be carried out immediately after obtaining composition B has been obtained. However, it is also envisaged to store composition B for a period of time as it was shown in the studies underlying the present invention that composition B has long-term stability (see Examples). For example, composition B may be stored for at least one month, such as for at least three months, such as for at least six months. In an embodiment, the composition is stored for one to three months. In alternative embodiment, composition B is stored for three to twelve months, such as for three to six months. Composition B may be stored at temperatures of between 2° C. and 25° C., such as at temperatures of 5° C. to 25° C. Typically, composition B is stored refrigerated at temperatures between 2° C. and 8° C., or under ambient conditions at temperatures between 17° C. and 23° C.

The reconstitution of composition B can be done in any aqueous (reconstitution) solution (e.g. an aqueous buffer) deemed appropriate. In the studies described in the Examples section, composition B was successfully reconstituted in the following aqueous reconstitution solutions:
1.5% or 200 mM glycine,
3% or 150 mM arginine-Cl,
6.4% or 200 mM arginine-glutamate,
5.8% or 150 mM magnesium-glutamate,
3% or 150 mM arginine-Cl and 1.5% or 200 mM glycine, and
3% or 150 mM arginine-CI and 5.8% or 150 mM magnesium-glutamate.

In an embodiment, the reconstitution solution is water, i.e. sterile water. Thus, the powder or micropellets are reconstituted in water.

In order to yield composition B, a volume of aqueous reconstitution buffer is used which achieves the desired antibody concentration of the antibody in composition I, i.e. a volume which yields composition I with the desired concentration of the antibody, such as a concentration of 300 mg/ml.

It is to be understood that reconstitution of the composition B in the aqueous reconstitution requires a certain reconstitution time. In an embodiment, the reconstitution is done for a time period which is sufficient to solve the antibody in the aqueous solution.

Advantageously, it has been shown that the reconstitution time for the spray freeze-dried composition as referred to herein is shorter than the reconstitution time for the spray dried or freeze-dried composition B.

In an embodiment of the provided method, the dried antibody composition B has been obtained by spray drying and, thus, is a spray dried powder. Typically, the spray dried powder is reconstituted in a reconstitution solution for a time period of at least four hours.

In an embodiment of the provided method, the dried antibody composition B has been obtained by freeze drying and, thus, is a freeze-dried powder. Typically, the freeze-dried powder is reconstituted in a reconstitution solution for a time period of 1.5 to 4.0 hours.

In an embodiment of the provided method, the dried antibody composition B has been obtained by spray freeze drying, and, thus, is in the form of spray freeze-dried micropellets. Typically, the spray freeze-dried micropellets are reconstituted in a reconstitution solution in a period of 1 to 2 hours.

The short reconstitution times allow for preparing the final composition I shortly before administering said composition. For example, composition I can be prepared by medical personnel, such as by medical personnel in a hospital. Short reconstitution times also allow for preparation of high concentration aqueous solution to be filled directly in vials or prefilled syringes. For example, composition I can be prepared by reconstitution composition B and filled in vials or pre-filled syringes in a manufacturing plant and marketed as a ready to use liquid product.

However, the present invention is not limited to the preparation of composition I shortly before its administration. Since composition I is stable, it can be prepared well in advance, e.g. by the manufacturer of the dried composition. Subsequently, composition I can be stored for an extended period. For example, composition B may be stored for at least one month, such as for at least three months, such as for at least six months under refrigerated or ambient storage conditions. In an embodiment, the composition is stored for one to three months. In alternative embodiment, composition I is stored for three to twelve months, such as for three to six months. Typically, composition B is stored refrigerated at temperatures between 2° C. and 8° C.

The definitions and explanations given herein above apply mutatis mutandis to the following.

Further provided is a container comprising the aqueous antibody composition I provided herein.

In an embodiment, the container is a prefilled syringe.

In alternative embodiment, the container is a vial.

In an alternative embodiment, the container is an autoinjector.

The container may comprise any volume of the aqueous antibody composition I deemed appropriate. In an embodiment, the container comprises about 1 to 4 ml of composition I, such as 1 to 2 ml of composition I. For example, the container may comprise about 1 ml, 2 ml, 3 ml or 4 ml of composition I. The volume may depend on the type the container. For example, a vial may comprise a volume of up to 50 ml, wherein a pre-filled syringe may comprise a volume of up to 2 ml.

Also provided is a kit comprising the container provided herein, and, optionally, a label or instructions for the administration and use of the aqueous antibody composition I or of the dried antibody composition B.

If the container comprises the dried antibody composition B, the kit may further comprise a reconstitution buffer. Typically, the kit comprises a volume of the reconstitution buffer which is sufficient to yield composition I.

Further provided is the use of the aqueous antibody composition I or of the dried antibody composition B provided herein for use in medicine.

Further provided is a method of producing a dried antibody composition B,
a) providing an aqueous composition A comprising
   a1) an antibody in a concentration as set forth in connection with the method of producing composition I, such as less than 200 mg/ml antibody, such as 10 to 190 mg/ml antibody, such as 20 to 170 mg/ml antibody, such as 50 to 150 mg/ml antibody, such as 80 to 120 mg/ml antibody based on the total volume of composition A.
   a2) at least surfactant,
   a3) at least one cryoprotectant, and
   a4) at least 0.4% (w/v) arginine, or a salt thereof, wherein the weight
   (w) is calculated as arginine, such as 0.4 to 3.5% (w/v) arginine wherein the ratio of the amount of a1) to the sum of the amounts of a3) and a4) is from 11:1 to 11:8, and
b) spray freeze drying the composition provided in step a) to give the dried antibody composition B.

In an embodiment, the dried antibody composition is in the form of micropellets.

Further provided is a dried antibody composition B, obtained or obtainable by the method of producing a dried antibody composition B.

Further provided is a dried antibody composition B being in the form of micropellets, the composition B comprising
a1) an antibody (as defined herein),
a2) at least one surfactant (as defined herein),
a3) at least one cryoprotectant (as defined herein), such as trehalose, and
a4) arginine, or a salt thereof (as defined herein),
wherein the ratio of the amount of a1) to the sum of the amounts of a3) and a4) is from 11:1 to 11:8, such as from 11:2 to 11:8, wherein the weight of a4 is calculated as the free amino acid.

In some embodiments, the ratio of the amount of a1) to the sum of the amounts of a3) and a4) is from 11:1 to 11:4, such as from 11:2 to 11:4.

Further provided is a dried antibody composition B being in the form of a powder, the composition B comprising
a1) an antibody (as defined herein),
a2) at least one surfactant (as defined herein),
a3) at least one cryoprotectant (as defined herein), such as trehalose, and
a4) arginine, or a salt thereof (as defined herein),
wherein the ratio of the amount of a1) to the sum of the amounts of a3) and a4) is from 11:1 to 11:8, such as from 11:2 to 11:8, wherein the weight of a4 is calculated as the free amino acid.

In some embodiments, the ratio of the amount of a1) to the sum of the amounts of a3) and a4) is from 11:1 to 11:4, such as from 11:2 to 11:4.

Further provided is a container comprising the dried antibody composition B provided herein. For example, the container may be a vial, a prefilled syringe, a cartridge, an ampule, or an autoinjector. In some embodiments, the container is a prefilled syringe or an autoinjector.

Also provided is the use of the dried antibody composition B provided herein for producing an aqueous antibody composition I comprising at least 200 mg/ml antibody, for example more than 200 mg/ml antibody.

Further provided is an aqueous antibody composition I as described herein. The composition may be used in medicine.

In an embodiment, the aqueous antibody composition I comprises 270 to 350 mg/ml antibody, such as 280 to 320 mg/ml antibody.

In a further embodiment, the aqueous antibody composition I comprises at least 250 mg/ml antibody, for example at least 300 mg/ml antibody.

In another embodiment of the provided aqueous antibody composition I comprises 230 mg/ml to 420 mg/ml antibody, such as 260 mg/ml to 400 mg/ml antibody.

In an embodiment, the aqueous antibody composition I comprises about 300 mg/ml antibody.

Typically, the aqueous antibody composition I has an opalescence below 40 NTU (Nephelometric Turbidity Units (NTU), such as an opalescence of below 25 NTU. For example, the opalescence may be below 15 NTU.

In an embodiment, the aqueous antibody composition I additionally comprises at least one surfactant, at least one cryoprotectant, such as trehalose, and arginine, or a salt thereof.

In some embodiments, the aqueous antibody composition I is obtained or obtainable by the method for producing an aqueous antibody composition I as provided herein.

Further, provided is an aqueous composition A as defined in connection with the method for producing an aqueous antibody composition I comprising at least 200 mg/ml antibody, said composition A comprising
a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
a2) at least one surfactant,
a3) at least one cryoprotectant, and
a4) at least 0.4% (w/v) arginine, or a salt thereof, wherein the weight (w) is calculated as arginine, based on the total volume of the aqueous composition A, such as 0.4 to 3.5% (w/v) arginine, wherein the ratio of the amount of a1) to the sum of the amounts of a3) and a4) is from 11:1 to 11:8.

The definitions and explanations made for composition A in connection with the method for producing an aqueous antibody composition apply mutatis mutandis.

### Embodiments

In the following, an overview of the provided embodiments is provided. The definitions and explanations given herein above apply mutatis mutandis to the following.

1. A method for producing an aqueous antibody composition I comprising at least 200 mg/ml antibody, wherein the method comprises the steps
a) providing an aqueous composition A comprising
   a1) an antibody in a concentration which is lower than the concentration of the antibody in the antibody composition I,
   a2) at least one surfactant,
   a3) at least one cryoprotectant,
   a4) at least 0.4% (w/v) arginine, or a salt thereof, wherein the weight
   (w) is calculated as arginine, such as 0.4 to 3.5% (w/v) arginine wherein the ratio of the amount of a1) to the sum of the amounts of a3) and a4) is from 11:1 to 11:8, and
b) drying the composition provided in step a) to yield an antibody composition B, and
c) reconstituting the composition dried in step b) to give the aqueous antibody composition I.

2. The method of embodiment 1, wherein the salt of arginine is arginine-CI, arginine-aspartate, arginine-glutamate, arginine-sulfate, arginine-acetate, arginine-succinate.

3. The method of embodiment 2, wherein the salt of arginine is arginine-Cl.

4. The method of any one of embodiments 1 to 3, wherein the at least one cryoprotectant is selected from the group of cryoprotectants consisting of trehalose, sucrose, sorbitol, glycerol and mannitol.

5. The method of embodiment 4, wherein the cryoprotectant is trehalose.

6. The method of any one of embodiments 1 to 5, wherein the ratio of the amount of a1) to the sum of the amounts of a3) and a4) is from 11:1 to 11:4, such as from 11:2 to 11:4,

7. The method of any one of embodiments 1 to 6, wherein the aqueous composition A comprises less than 200 mg/ml antibody, such as 10 to 190 mg/ml antibody, such as 20 to 170 mg/ml antibody, such as 50 to 150 mg/ml antibody, such as 80 to 120 mg/ml antibody.

8. The method of any one of embodiments 1 to 7, wherein composition I comprises more than 200 mg/ml antibody, for example 230 mg/ml to 420 mg/ml antibody, such as 260 mg/ml to 400 mg/ml antibody.

9. The method of embodiment 8, wherein composition I comprises 270 to 350 mg/ml antibody, such as 280 to 320 mg/ml antibody, such as 300 mg/ml antibody.

10. The method of any one of embodiments 1 to 9, wherein the antibody is a monoclonal antibody.

11. The method of any one of embodiments 1 to 10, wherein the at least surfactant is a polysorbate, such as polysorbate 20 or 80, or a polaxamer, such as polaxamer 188.

12. The method of any one of embodiments 1 to 11, wherein composition A further comprises a5) a buffer, for example a histidine, citrate, acetate, phosphate, Tris, succinate or glycine buffer.

13. The method of any one of embodiments 1 to 12, wherein the pH value of the composition A is about 5.0 to 8.0, such as about 5.5 to 7.

14. The method of any one of embodiments 1 to 13, wherein the antibody composition B is in the form of a powder or of micropellets.

15. The method of any one of embodiments 1 to 14, wherein step b) is carried out by freeze drying or spray drying to give a freeze-dried or spray dried powder.

16. The method of embodiment 15, wherein the freeze-dried powder is reconstituted in a reconstitution solution for a time period of 1.5 to 4.0 hours, or wherein the spray dried powder is reconstituted in a reconstitution solution for a time period of at least four hours.

17. The method of any one of embodiments 1 to 14, wherein step b) is carried out by spray freeze drying to give spray freeze-dried micropellets.

18. The method of embodiment 17, wherein the spray freeze-dried micropellets are reconstituted in a reconstitution solution in a period of 1 to 2 hours.

19. The method of any one of embodiments 1 to 18, wherein the aqueous antibody composition I is a clear and transparent solution.

20. An aqueous antibody composition I obtained or obtainable by the method of any one of embodiments 1 to 19.

21. A container comprising the aqueous antibody composition I of embodiment 20.

22. The container of embodiment 21, wherein the container is a prefilled syringe, a vial, a cartridge, an ampule or an autoinjector.

23. A kit comprising the container of embodiment 21 or 22, and a label or instructions for the administration and use of the aqueous antibody composition I.

24. The aqueous antibody composition I of embodiment 21 for use in medicine.

25. A method of producing a dried antibody composition B,
a) providing an aqueous composition A comprising
   a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
   a2) at least surfactant,
   a3) at least one cryoprotectant, and
   a4) at least 0.4% (w/v) arginine, or a salt thereof, wherein the weight
   (w) is calculated as arginine, such as 0.4 to 3.5% (w/v) arginine wherein the ratio of the amount of a1) to the sum of the amounts of a3) and a4) is from 11:1 to 11:8, and
b) spray freeze drying the composition provided in step a)
to give the dried antibody composition B.

26. The method of embodiment 25, wherein the dried antibody composition is in the form of micropellets.

27. Dried antibody composition B, obtained or obtainable by the method of embodiment 25 or 26.

28. A container comprising the dried antibody composition B of embodiment 27.

29. Use of the dried antibody composition of embodiment 26 or 27 for producing an aqueous antibody composition I comprising at least 200 mg/ml antibody.

30. An aqueous antibody composition I comprising at least 200 mg/ml antibody.

31. Aqueous antibody composition I according to embodiment 30, comprising 270 to 350 mg/ml antibody, such as 280 to 320 mg/ml antibody, such as 300 mg/ml antibody.

32. Aqueous antibody composition I according to embodiment 30, comprising 230 mg/ml to 420 mg/ml antibody, such as 260 mg/ml to 400 mg/ml antibody.

33. Aqueous antibody composition I according to any one of embodiments 30 to 32, wherein the composition is a clear and transparent solution.

34. Aqueous antibody composition I according to any one of embodiments 30 to 33, having an opalescence below 40 NTU (Nephelometric Turbidity Units (NTU), such as an opalescence of below 25 NTU or below 15 NTU.

35. Aqueous antibody composition I according to any one of embodiments 30 to 34 comprising at least one surfactant, at least one cryoprotectant, and arginine, or a salt thereof.

36. Aqueous antibody composition I according to embodiment 35, wherein the at least one cryoprotectant is trehalose.

37. Aqueous antibody composition I according to embodiment 35 or 36, wherein the at least one surfactant is a polysorbate, such as polysorbate 20 or 80, or a polaxamer, such as polaxamer 188.

38. Aqueous antibody composition I according to any one of embodiments 30 to 37, comprising histidine.

39. Aqueous antibody composition I according to any one of embodiments 30 to 38, additionally comprising glycine and/or magnesium-glutamate.

40. Dried antibody composition B being in the form of micropellets, the composition B comprising
a1) an antibody (as defined herein),
a2) at least one surfactant (as defined herein),
a3) at least one cryoprotectant (as defined herein), such as trehalose, and
a4) arginine, or a salt thereof (as defined herein),
wherein the ratio of the amount of a1) to the sum of the amounts of a3) and a4) is from 11:1 to 11:8, such as from 11:2 to 11:8, wherein the weight of a4 is calculated as the free amino acid.

41. Dried antibody composition B being in the form of a powder, the composition B comprising
a1) an antibody (as defined herein),
a2) at least one surfactant (as defined herein),
a3) at least one cryoprotectant (as defined herein), such as trehalose, and
a4) arginine, or a salt thereof (as defined herein),
wherein the ratio of the amount of a1) to the sum of the amounts of a3) and a4) is from 11:1 to 11:8, such as from 11:2 to 11:8, wherein the weight of a4 is calculated as the free amino acid.

42. Use of the dried antibody composition of embodiment 40 or 41 for producing an aqueous antibody composition I comprising at least 200 mg/ml antibody.

43. An aqueous composition A comprising
a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
a2) at least one surfactant,
a3) at least one cryoprotectant, and
a4) at least 0.4% (w/v) arginine, or a salt thereof, wherein the weight (w) is calculated as arginine, based on the total volume of the aqueous composition A, such as 0.4 to 3.5% (w/v) arginine, wherein the ratio of the amount of a1) to the sum of the amounts of a3) and a4) is from 11:1 to 11:8.

44. The aqueous composition A of embodiment 43, wherein the salt of arginine is arginine-Cl, arginine-aspartate, arginine-glutamate, arginine-sulfate, arginine-acetate, arginine-succinate.

45. The aqueous composition A of embodiment 44, wherein the salt of arginine is arginine-Cl.

46. The aqueous composition A of any one of embodiments 43 to 45, wherein the at least one cryoprotectant is selected from the group of cryoprotectants consisting of trehalose, sucrose, sorbitol, glycerol and mannitol.

47. The aqueous composition A of embodiment 46, wherein the cryoprotectant is trehalose.

48. The aqueous composition A of any one of embodiments 43 to 47, wherein the ratio of the amount of a1) to the sum of the amounts of a3) and a4) is from 11:1 to 11:4, such as from 11:2 to 11:4,

49. The aqueous composition A of any one of embodiments 43 to 48, wherein the aqueous composition A comprises less than 200 mg/ml antibody, such as 10 to 190 mg/ml antibody, such as 20 to 170 mg/ml antibody, such as 50 to 150 mg/ml antibody, such as 80 to 120 mg/ml antibody.

50. The aqueous composition A of any one of embodiments 43 to 49, wherein the antibody is a monoclonal antibody.

51. The aqueous composition A of any one of embodiments 43 to 50, wherein the at least surfactant is a polysorbate, such as polysorbate 20 or 80, or a polaxamer, such as polaxamer 188.

52. The aqueous composition A of any one of embodiments 43 to 51, wherein composition A further comprises a5) a buffer, for example a histidine, citrate, acetate, phosphate, Tris, succinate or glycine buffer.

53. The aqueous composition A of any one of embodiments 43 to 52, wherein the pH value of the composition A is about 5.0 to 8.0, such as about 5.5 to 7.

54. Use the aqueous antibody composition A as defined in any one of claims 43 to 53 for producing an aqueous antibody composition I comprising at least 200 mg/ml antibody.

All references cited throughout this specification are herewith incorporated with respect to the disclosure content specifically referred to above as well as in their entireties.

The present invention is now further described by reference to the following Examples, which are intended to illustrate, and not to limit, the scope of the present invention.

### EXAMPLES

### Materials and Methods

### Physical Stability by Size Exclusion Chromatography (SEC)

Physical stability was assessed by periodically measuring the amount of aggregates generated in monoclonal antibody formulations. Samples were measured by eluting ~100 μg of total monoclonal antibody off of a TSK-Gel G3000SWXL analytical column (Tosoh Co.) with 40 mM Phosphate, 150 mM Sodium Chloride at pH 7.2 (detection wavelength: 280 nm). Aggregates were designated as high molecular weight species (HMWS) that eluted faster than non-aggregated mAb monomers; HMWS % was calculated by dividing the amount of aggregate generated by the sum of total aggregated+non-aggregated monoclonal antibody molecules.

### SoloVPE for Protein Concentration

Total protein concentration was determined by measuring the absorbance at 280 nm using Lunatic instrument (Unchained Labs). Samples for the powder arm were measured without any dilution and samples for the liquid arm were measured after a 10 to 15-fold dilution in the formulation buffer. Two measurements were performed on each sample of 3 μL and the average value was reported. Molar extinction coefficient that was antibody-specific was used for concentration calculation. The results were considered comparable when the difference was equal or less than 10%.

### Opalescence

Samples were analyzed according to the protocol established in Kingsbury J S et al., J Pharm Sci. 2021 September; 110(9):3176-3182. doi: 10.1016/j.xphs.2021.05.005. Epub 2021 May 15. PMID: 34004217. Briefly, samples were analyzed by u-nephelometry using a “de-tuned” static light scattering channel of a Wyatt DynaPro NanoStar light scattering instrument. Samples were loaded in 10-μl volumes into NanoStar disposable cuvettes. The instrument was set to acquire data with the laser power diminished to 1%. The static scattering detector voltage was recorded and calibrated against a set of polymer bead turbidity standards of 1, 5, 10, 20, 40 and 80 NTU.

### Spray Freeze Drying

Spray freeze drying was performed using process parameters similar to ones described in Clenet et al. (Clénet D, Hourquet V, Woinet B, Ponceblanc H, Vangelisti M. A spray freeze dried micropellet based formulation proof-of-concept for a yellow fever vaccine candidate. Eur J Pharm Biopharm. 2019 September; 142:334-343). High level process parameters used for spray freeze drying:

Prilling

| Vibration frequency | 4000 | Hz |
|---|---|---|
| Flow Rate | 35 tpm (~20 L/min) | |
| Voltage | 125 | mV |
| Deflection pressure | 0.20 | bar |
| Temperature at the top | −100° | C. |
| Temperature at the bottom | −70° | C. |

Lyophilization
2a. Loading

| Pre-cooling of the shelves | Yes | |
|---|---|---|
| Shelf temperature | −50° | C. |
| Time maintained at shelf temperature | 30 | min |

2b. Condenser cooling and void setting

| | | |
|---|---|---|
| Pressure | 50 | μbar |
| Condenser temperature | −45° | C. |

2c. Primary drying

| | | |
|---|---|---|
| Shelf temperature | −30° | C. |
| Ramp duration | 120 | min |
| Time maintained at shelf temperature | 180 | min |
| Pressure | 50 | μbar |

2d. Secondary drying

| | | |
|---|---|---|
| Shelf temperature | 20° | C. |
| Ramp duration | 600 | min |
| Time maintained at shelf temperature | 323 | min |
| Pressure | 50 | μbar |

### Spray Drying

### High Level Process Parameters Used for Spray Drying:

| | |
|---|---|
| Temperature (° C.) | 90 |
| Temperature chamber out (° C.) | ~54 |
| Temperature before cycle (° C.) | 40 |
| Airflow (m³/min) | 0.4 |
| Cooling air (L/min) | 300 |
| Nozzle air (L/min) | 8 |
| Absolute humidity (g/m³) | 10 |

### Example 1: Feasibility of Achieving High Concentration Formulation

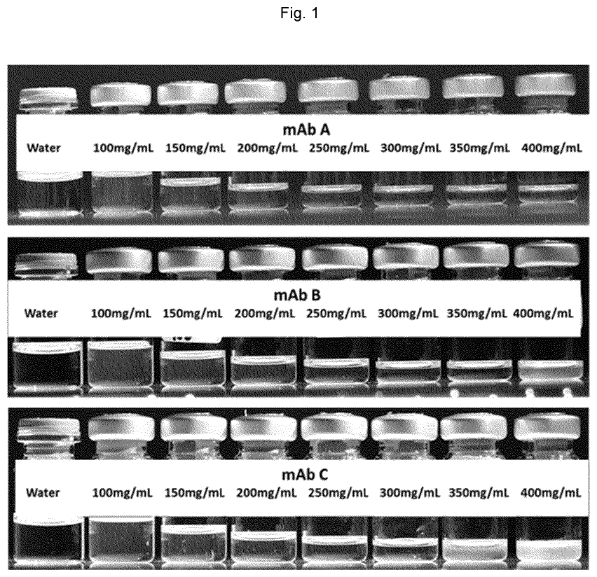

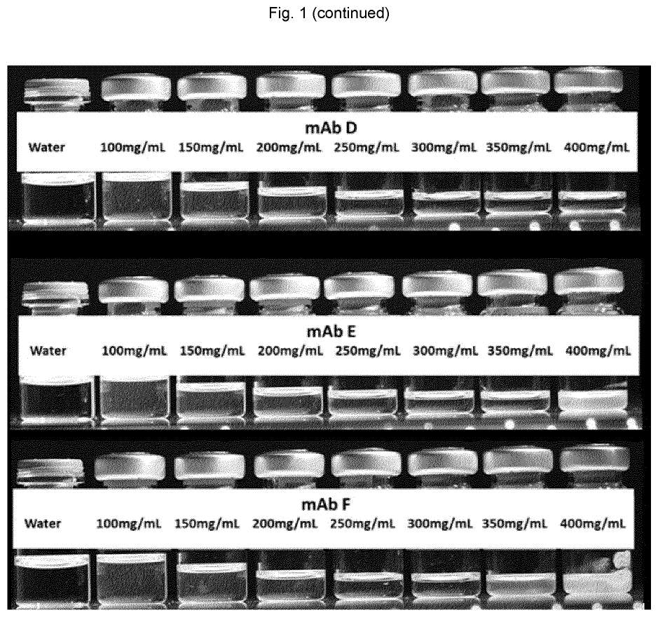

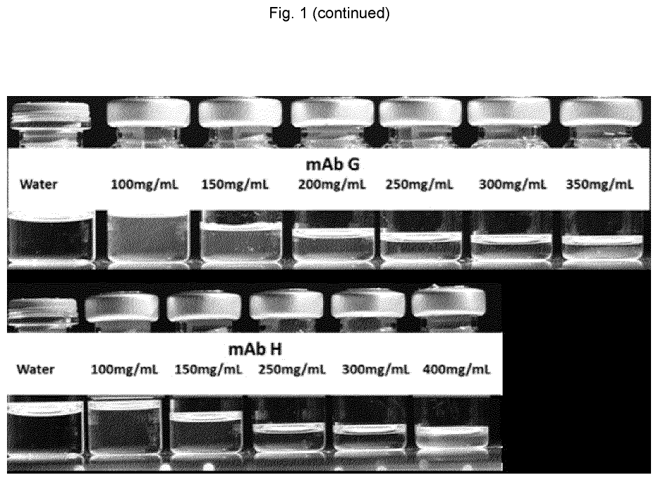

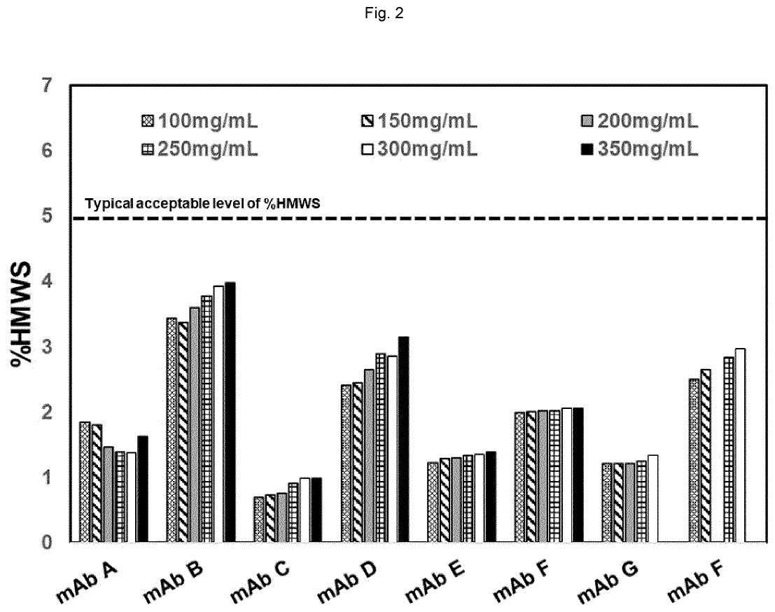

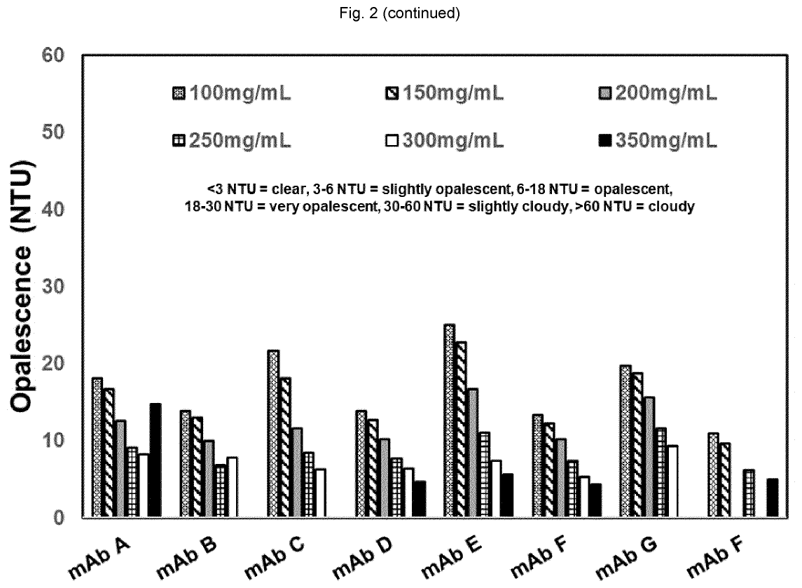

Eight monoclonal antibodies (labeled “mAb A”, “mAb B”, “mAb C”, “mAb D”, “mAb E”, “mAb F”, “mAb G” and “mAb H”) were used for this feasibility assessment. While mAb's A, B, and G were IgG4 sub-type, others belonged to IgG1 sub-type. All antibodies were formulated in a starting aqueous composition A consisting of 100 mg/mL antibody, 10 mM histidine, 2% trehalose, 0.05% Polysorbate 80 (ratio of the amount of antibody to amount of trehalose is 10:2 w/w). pH of each composition was selected based on the isoelectric point and formulation screen studies. Each composition A was freeze-dried to yield intermediate dried antibody powder (composition B). This intermediate dried antibody powder was reconstituted with aqueous solution containing 1.75% or 100 mM arginine resulting in an aqueous antibody composition (composition I). Reconstitution volumes were chosen to achieve aqueous antibody composition I of 100, 150, 200, 250, 300, 350, and 400 mg/mL antibody concentration. Target protein concentrations were achieved for all eight antibodies, as measured by SoloVPE. Clear and transparent solution was generally achieved for all concentrations and antibodies tested (FIG. 1). Clear and transparent solution was achieved for all eight antibodies up to 300 mg/mL. With the exception of mAb C, clear and transparent solution was achieved for seven antibodies up to 350 mg/mL. Clear and transparent solution up to 400 mg/mL was achieved for mAb D, mAb G, and mAb H. Increase in antibody concentration is typically associated with increase in HMWS due to molecular crowding phenomena. However, in this example, increase in antibody concentration from 100 to 350 mg/mL is generally associated with a marginal increase in HMWS (less than 1%), and modest decrease in solution opalescence (FIG. 2). HMWS levels below 5% and less than 1% change in HMWS upon increase in concentration are both robust indicators of physical stability of antibody composition I with at least 200 mg/mL antibody concentration. Opalescence of all antibody compositions I with at least 200 mg/mL antibody concentration is less than 20 NTU, another robust indicator of optimal solution properties. Antibody composition I between and including 200 to 350 mg/mL provide optimal properties such as clear and transparent solution, low HMWS, and opalescence.

### Example 2: Optimal Composition for Aqueous Composition A

Four monoclonal antibodies (mAb A, B, D, and E) and eleven compositions were evaluated in this example. An overview is provided in Table 1.

TABLE 1
Description of the 11 compositions evaluated
for Example 2 using mAb A, B, D, and E

| mAb:(trehalose + | Formulation | | |
|---|---|---|---|
| arginine) w/w ratio* | ID | Formulation | |
| 11:1 | F1 | 100 mg/mL | 0.5% trehalose, 0.45% Arginine |
| | F2 | mAb, | 0.9% Arginine |
| 11:2 | F3 | 0.05% | 2% trehalose |
| | F4 | Polysorbate | 1.5% trehalose, 0.45% Arginine |
| | F5 | 80, 10 mM | 1% trehalose, 0.9% Arginine |
| | F6 | histidine | 1.75% Arginine |
| 11:4 | F7 | | 4% trehalose |
| | F8 | | 3.5% trehalose, 0.45% Arginine |
| | F9 | | 3% trehalose, 0.9% Arginine |
| | F10 | | 3.5% Arginine |
| 11:8 | F11 | | 4% trehalose, 3.5% Arginine |

All antibodies were formulated in a starting aqueous composition A consisting of 100 mg/mL antibody, 10 mM histidine, 0.05% Polysorbate 80, 0.5 to 4% trehalose (molecular weight 343), 0.45 to 3.5% arginine (molecular weight 174.2 g/mol). Trehalose is used as cryoprotectant. This represented a ratio of the amount of antibody to the sum of the amounts of cryoprotectant trehalose and arginine free base from 11:1 to 11:8% and covered different combinations of trehalose and arginine between 0.5 to 4% and 0.45 to 3.5% respectively. pH of these formulations were antibody-specific and chosen during formulation screening to provide optimal stability. Antibodies in starting aqueous composition A were freeze-dried to yield intermediate dried antibody powder (composition B). The results are shown in Table 2.

TABLE 2
Assessment of physical stability of composition A during freeze drying

| mAb:(trehalose + | | ΔHMWS during lyophilization | | | |
|---|---|---|---|---|---|
| arginine) w/w | Composition | (HMWS after freeze drying − HMWS before freeze drying) | | | |
| ratio | ID | mAb A | mAb B | mAb D | mAb E |
| 11:1 | F1 | 0.26 | 0.58 | 0.30 | 0.29 |
| | F2 | 0.21 | 0.47 | 0.29 | 0.24 |
| 11:2 | F3 | 0.46 | 0.62 | 0.62 | 0.41 |
| | F4 | 0.15 | 0.48 | 0.17 | 0.20 |
| | F5 | 0.20 | 0.34 | 0.18 | 0.15 |
| | F6 | 0.17 | 0.33 | 0.20 | 0.16 |
| 11:4 | F7 | 0.15 | 0.63 | 0.03 | 0.12 |
| | F8 | 0.15 | 0.16 | 0.10 | 0.10 |
| | F9 | 0.13 | 0.32 | 0.01 | 0.48 |
| | F10 | 0.07 | −0.08 | 0.09 | 0.04 |
| 11:8 | F11 | 0.05 | −0.04 | 0.10 | 0.04 |

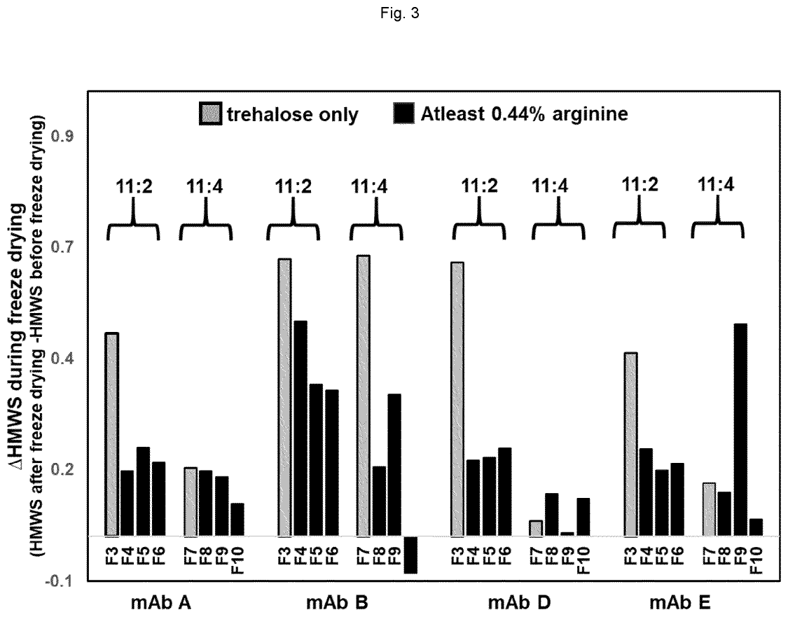

As shown in Table 2, there was only a marginal increase in HMWS (<0.7% in all cases) in HMWS during freeze drying for all antibodies and compositions, generally indicating that all compositions between 11:1 to 11:8 w/w of antibody/trehalose+arginine-protect the antibody from degradation during freeze drying. Increase in HMWS of 0.7% is generally a robust indicator of the stability of composition A during freeze drying. Data also indicated that higher levels of trehalose and arginine such as 11:2 to 11:8 compositions generally favored antibody stability. We also observed that the starting aqueous composition A containing at least 0.45% arginine (MW 174.2 g/mol) (black bars in FIG. 3) generally had lower increase in HMWS during freeze drying relative to trehalose only (gray bars in FIG. 3) compositions. Evaluation of short-term storage stability of intermediate freeze-dried antibody (composition B) at 25° C. indicated modest increase (generally <1% for mAb A, D, E and generally <2% for mAb B) in HMWS at 1 month storage at 25° C. relative to initial timepoint, generally suggesting that all compositions provide protection during storage of the intermediate freeze-dried antibody (Table 3).

TABLE 3
Assessment of physical stability of composition B upon storage at 25° C. for 1 month

| | | ΔHMWS after storage of intermediate dried powder | | | |
|---|---|---|---|---|---|
| mAb:(trehalose + | | (HMWS after storage of freeze-dried powder at 25° C. for | | | |
| arginine) w/w | Composition | 1 month − HMWS immediately after freeze drying) | | | |
| ratio | ID | mAb A | mAb B | mAb D | mAb E |
| 11:1 | F1 | 1.1 | 1.9 | 1.3 | 1.0 |
| | F2 | 0.9 | 1.6 | 1.1 | 0.8 |
| 11:2 | F4 | 0.9 | 1.9 | 1.0 | 0.8 |
| | F5 | 0.7 | 1.3 | 0.8 | 0.6 |
| 11:4 | F8 | 0.5 | 1.0 | 0.7 | 0.4 |
| | F10 | 0.2 | 0.2 | 0.3 | 0.0 |
| 11:8 | F11 | 0.1 | 0.1 | 0.2 | 0.0 |

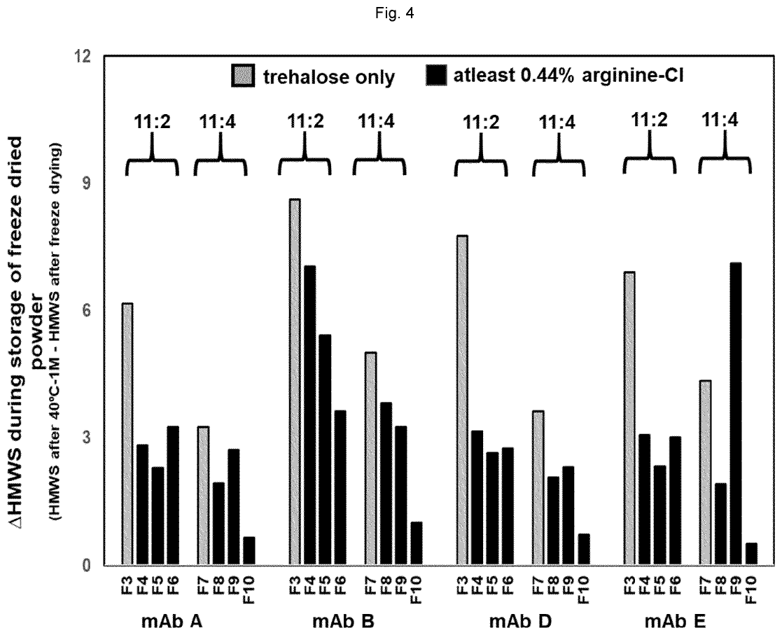

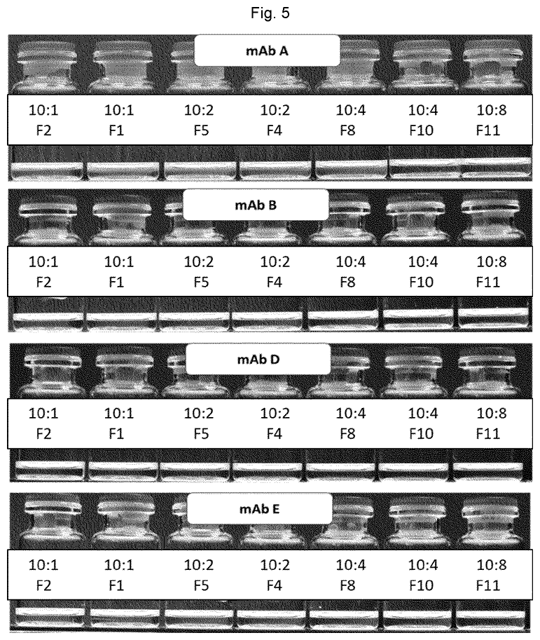

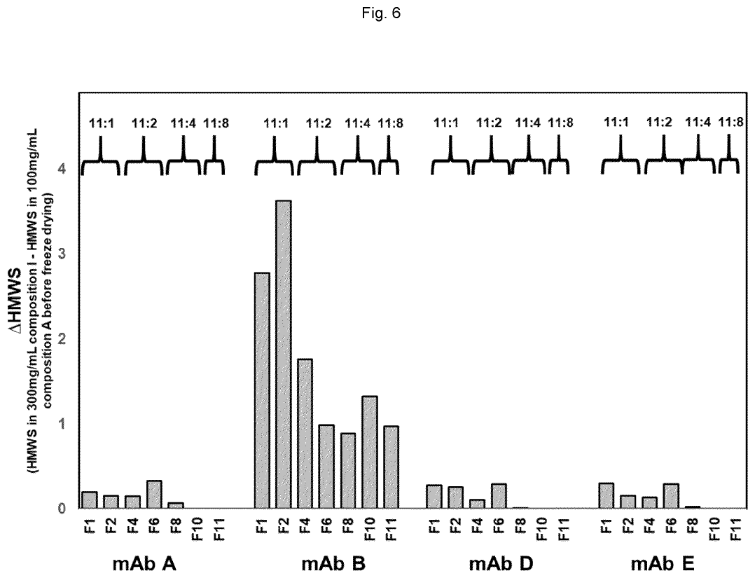

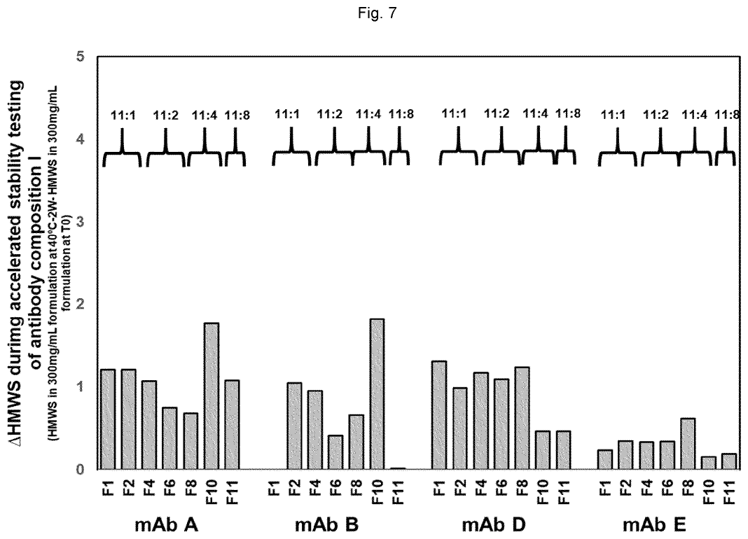

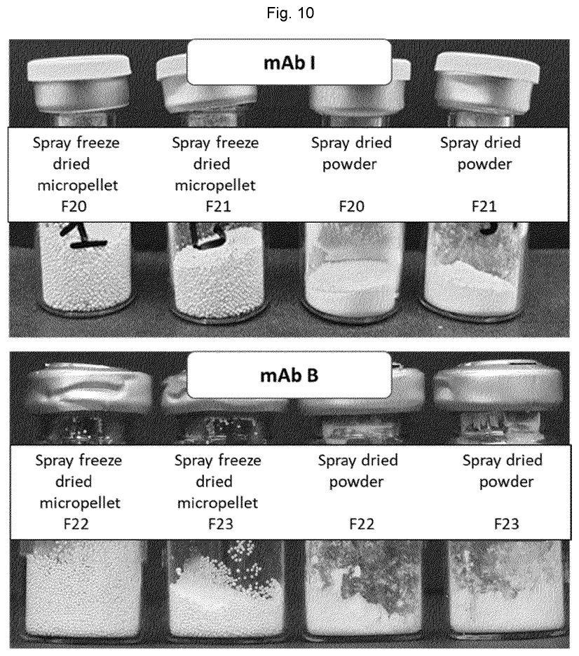

Data also indicated that higher levels of trehalose and arginine such as 11:2 to 11:8 compositions generally favored antibody stability during storage at 25° C. Evaluation of accelerated stability of intermediate freeze-dried antibody (composition B) at 40° C. indicated that compositions containing at least 0.45% arginine (MW 174.2 g/mol) (black bars in FIG. 4) generally had lower increase in HMWS relative to trehalose only (grey bars in FIG. 4) compositions. Following this, the intermediate dried antibody powder (composition B) was reconstituted with Water for Injection (WFI). Reconstitution volumes were chosen to achieve aqueous antibody composition I of 300 mg/mL antibody concentration (composition I). Clear and transparent aqueous solution with target protein concentration of 300 mg/mL were achieved for all antibodies and compositions shown in FIG. 5. Comparison of HMWS in 300 mg/ml antibody composition I relative to HMWS in 100 mg/mL starting aqueous antibody composition A, both having the same antibody to trehalose, arginine weight ratio, generally indicates marginal increase in HMWS as a result of three-fold increase in antibody concentration. Change in HMWS between antibody composition I and antibody composition A was less than 0.5% for mAb A, D, and E. mAb B exhibited high increase in HMWS (less than 4%) but this was generally mitigated at 11:2 to 11:8 w/w (FIG. 6), where change in HMWS was generally less than 2%. Evaluation of accelerated stability of 300 mg/mL antibody composition I indicated modest increase (<2%) in HMWS at 2 weeks at 40° C. relative to initial timepoint, generally suggesting that all compositions provide protection to the antibody in composition I (FIG. 10). 40° C. represents a standard condition used for accelerated testing of biologics and is not the intended storage temperature for composition I. An increase of <2% in HMWS at 2 weeks at 40° C. is considered a small change generally indicative of high physical stability.

### Example 3: Optimal Reconstitution Solution for Intermediate Dried Antibody Powder

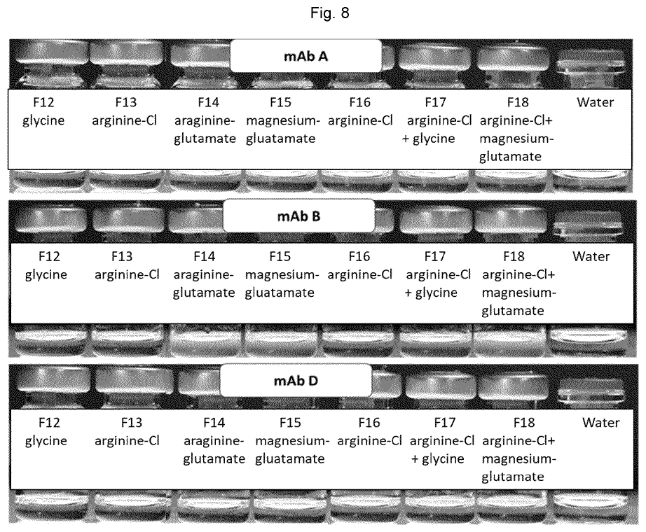

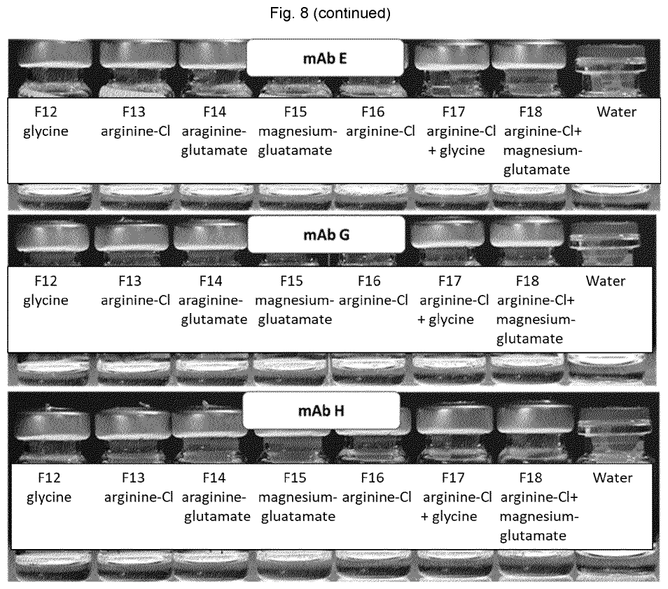

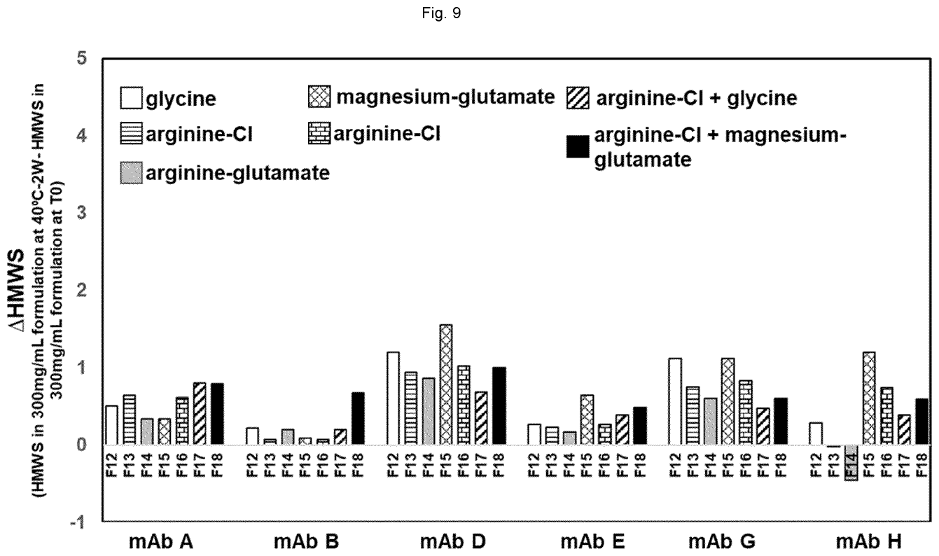

Six monoclonal antibodies (labeled mAb A, B, D, E, G, and H) were used for this assessment. All antibodies were either formulated in a starting aqueous composition A consisting of 100 mg/mL antibody, 10 mM histidine, 2% trehalose, 0.05% Polysorbate 80 (Formulations F12 to F15), which represents a ~11:2 w/w or 100 mg/mL antibody, 10 mM histidine, 3% trehalose, 0.05% Polysorbate 80 (Formulation 16 to Formulation 18), which represents a ~11:3 w/w. Antibodies in starting aqueous composition A were freeze-dried to yield intermediate dried antibody powder (composition B). Following this, dried antibody powder was reconstituted with 1.5% or 200 mM glycine (F12), 3% or 150 mM arginine-Cl (F13), 6.4% or 200 mM arginine-glutamate (F14), 5.8% or 150 mM magnesium-glutamate (F15), 3% or 150 mM arginine-CI (F16), 3% or 150 mM arginine-Cl and 1.5% or 200 mM glycine (F17), or 3% or 150 mM arginine-CI and 5.8% or 150 mM magnesium-glutamate (F17). Reconstitution volumes were chosen to achieve aqueous antibody composition I of 300 mg/mL antibody concentration. Clear and transparent aqueous solution with target antibody concentration of 300 mg/mL were achieved for all antibodies and compositions (FIG. 8). Evaluation of accelerated stability of 300 mg/ml antibody composition I indicated modest increase (<2%) in HMWS after 2 weeks at 40° C. relative to initial timepoint, generally suggesting that all compositions provide protection to the antibody in composition I (FIG. 9). 40° C. represents a standard condition used for accelerated testing of biologics and is not the intended storage temperature for composition I. An increase of <2% in HMWS at 2 weeks at 40° C. is considered a small change generally indicative of high physical stability. These changes in HMWS are similar to observations in FIG. 4, which involved trehalose and arginine-CI. This may generally suggest that reconstitution solutions containing glycine, arginine-CI, guanidinium-CI, arginine-glutamate, and magnesium-glutamate are effective in providing protection to the antibody in composition I.

### Example 4: Evaluation of Spray Drying and Spray Freeze Drying to Generate Intermediate Dried Antibody Powder

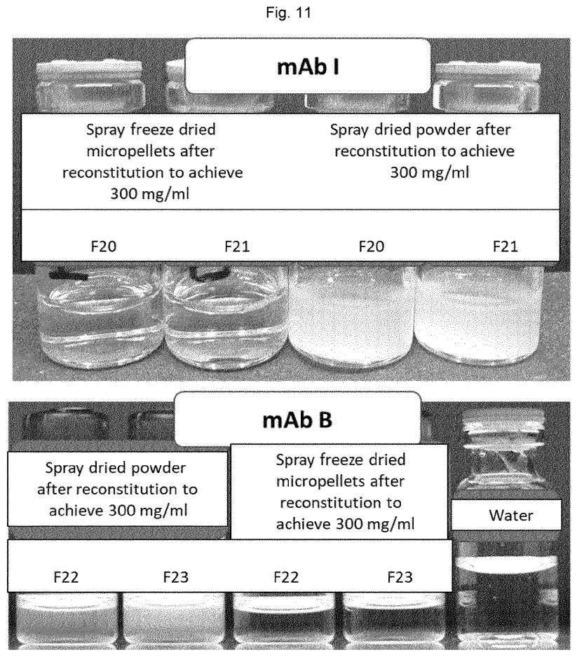

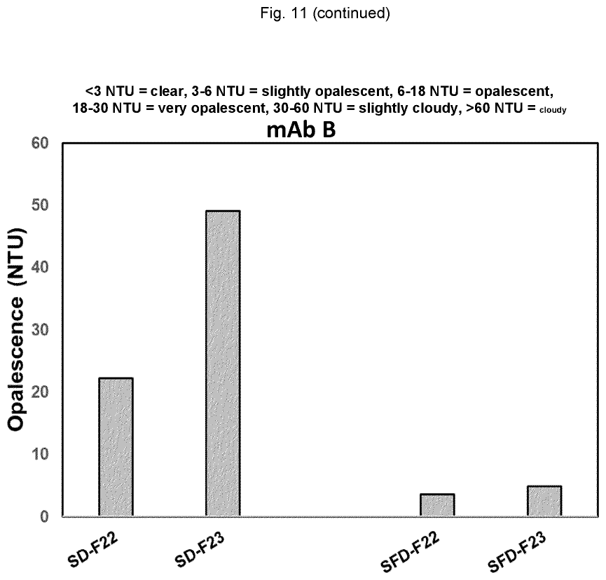

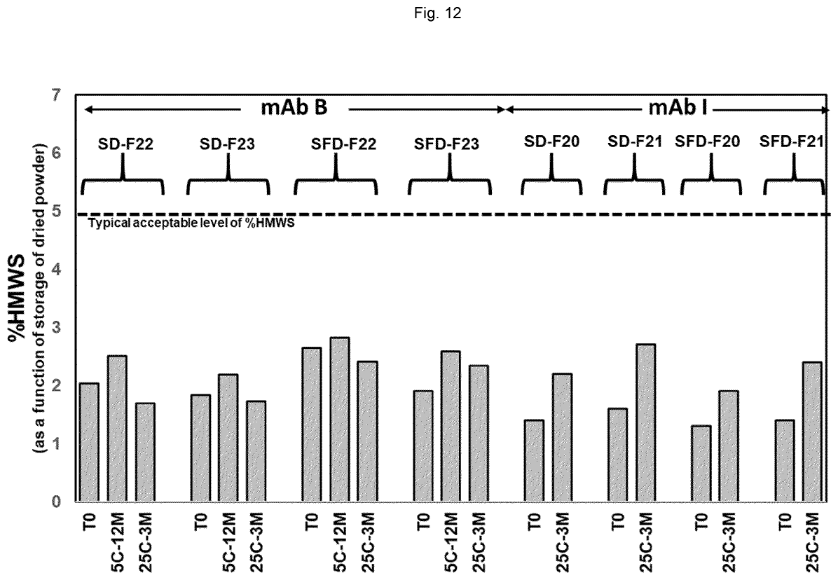

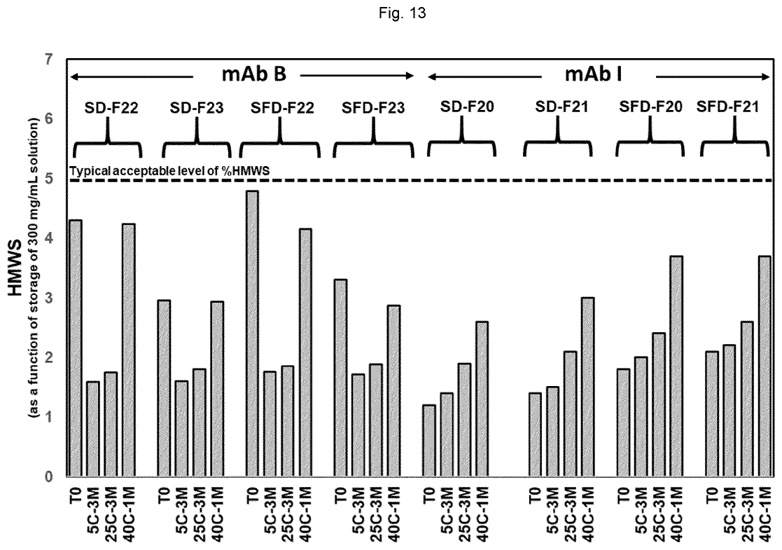

Two monoclonal antibodies (labeled mAb B and I) and two compositions for each antibody (F20 and F21 for mAb I, F22 and F23 for mAb B) were used for this assessment. Starting aqueous composition A of mAb B comprised of (F22) 100 mg/mL antibody, 0.06% Polysorbate 80, 1.75% arginine (representing ~11:2 w/w of antibody:trehalose+arginine) and (F23) 100 mg/mL antibody, 0.06% Polysorbate 80, 2% trehalose, 0.9% arginine (representing ~11:3 w/w of antibody:trehalose+arginine). Starting aqueous composition A of mAb I comprised of (F20) 100 mg/mL antibody, 10 mM histidine, 0.06% Polysorbate 80, 3% trehalose, 0.9% arginine (representing ~11:4 w/w) and (F21) 100 mg/mL antibody, 10 mM histidine, 0.06% Polysorbate 80, 2% trehalose, 0.45% arginine (representing ~11:2.5 w/w). Both antibodies in their respective starting composition A's were spray dried and spray freeze-dried to yield intermediate dried antibody powder (composition B). As shown in FIG. 10, spray drying resulted in loose dried powder and spray freeze drying resulted in micropellets. Intermediate dried antibody powder (composition B) from both drying techniques were reconstituted with Water for Injection. Reconstitution volumes were chosen to achieve aqueous antibody composition I of 300 mg/mL antibody concentration. Clear and transparent aqueous solution with target antibody concentration of 300 mg/mL were achieved for both antibodies (mAb B and I) and compositions (F22 and F23 for mAb B and F20 and F21 for mAb I) following reconstitution of spray freeze-dried micropellets (FIG. 11). Opalescence of resulting solution was <5 NTU for both compositions of mAb B generally indicating acceptable solution properties. Reconstitution times for reconstituting micropellets were generally 1 to 2 hours. While antibody concentrations of 300 mg/mL were achieved following reconstitution of spray dried powder, reconstitution times were generally greater than 4 hours, and the resulting solution generally had high turbidity (>20 NTU) and generally lacked clarity and transparency. Evaluation of short and long-term storage stability of the intermediate spray dried powder and spray freeze-dried micropellets (composition B) generally indicate no significant increase in HMWS following short-term storage at 25° C. or long-term storage at 5° C. (FIG. 12). Evaluation of short storage and accelerated stability of the 300 mg/ml composition I resulting from reconstitution of spray dried powder and spray freeze-dried micropellets generally indicate no significant increase in HMWS following short-term storage for 3 months at 5 or 25° C. or 1 month at 40° C. (FIG. 13).

1. A method for producing an aqueous antibody composition I comprising at least 200 mg/ml antibody, wherein the method comprises the steps
a) providing an aqueous composition A comprising
a1) an antibody in a concentration which is lower than the concentration of the antibody in the antibody composition I,
a2) at least one surfactant,
a3) at least one cryoprotectant,
a4) at least 0.4% (w/v) arginine, or a salt thereof, wherein the weight (w) is calculated as arginine, such as 0.4 to 3.5% (w/v) arginine
wherein the ratio of the amount of a1) to the sum of the amounts of a3) and a4) is from 11:1 to 11:8, and
b) drying the composition provided in step a) to yield an antibody composition B, and
c) reconstituting the composition dried in step b)
to give the aqueous antibody composition I.

2. The method of claim 1, wherein the salt of arginine is arginine-Cl, arginine-aspartate, arginine-glutamate, arginine-sulfate, arginine-acetate, arginine-succinate.

3. The method of claim 1, wherein the at least one cryoprotectant is selected from the group of cryoprotectants consisting of trehalose, sucrose, sorbitol, glycerol and mannitol.

4. The method of claim 1, wherein the ratio of the amount of a1) to the sum of the amounts of a3) and a4) is from 11:1 to 11:4, such as from 11:2 to 11:4.

5. The method of claim 1, wherein the aqueous composition A comprises less than 200 mg/ml antibody, such as 10 to 190 mg/ml antibody, such as 20 to 170 mg/ml antibody, such as 50 to 150 mg/ml antibody, such as 80 to 120 mg/ml antibody.

6. The method of claim 1, wherein composition I comprises 230 mg/ml to 420 mg/ml antibody, such as 260 mg/ml to 400 mg/ml antibody, or
wherein composition I comprises 270 to 350 mg/ml antibody, such as 280 to 320 mg/ml antibody, such as 300 mg/ml antibody.

7. The method of claim 1, wherein
a) the antibody is a monoclonal antibody,
b) the at least surfactant is a polysorbate, such as polysorbate 20 or 80, or a polaxamer, such as polaxamer 188,
c) the pH of the composition A is about 5.0 to 8.0, such as about 5.5 to 7, and/or
d) composition A further comprises a5) a buffer, for example a histidine, citrate, acetate, phosphate, Tris, succinate or glycine buffer.

8. The method of claim 1, wherein the antibody composition B is in the form of a powder or of micropellets.

9. The method of claim 1, wherein step b) is carried out by freeze drying or spray drying to give a freeze-dried or spray dried powder, and optionally wherein the freeze-dried powder is reconstituted in a reconstitution solution for a time period of 1.5 to 4.0 hours, or wherein the spray dried powder is reconstituted in a reconstitution solution for a time period of at least four hours.

10. The method of claim 1, wherein step b) is carried out by spray freeze drying to give spray freeze-dried micropellets.

11. The method of claim 10, wherein in step c) the spray freeze-dried micropellets are reconstituted in a reconstitution solution in a period of 1 to 2 hours.

12. An aqueous antibody composition I obtained or obtainable by the method of claim 1.

13. A container comprising the aqueous antibody composition I of claim 12, e.g., wherein the container is a prefilled syringe, a vial, a cartridge, an ampule or an autoinjector.

14. A method of producing a dried antibody composition B,
a) providing an aqueous composition A comprising
a1) less than 200 mg/ml antibody, such as 10 to 190 mg/ml antibody, such as 20 to 170 mg/ml antibody, such as 50 to 150 mg/ml antibody, such as 80 to 120 mg/ml antibody,
a2) at least surfactant,
a3) at least one cryoprotectant, and
a4) at least 0.4% (w/v) arginine, or a salt thereof, wherein the weight (w) is calculated as arginine, such as 0.4 to 3.5% (w/v) arginine
wherein the ratio of the amount of a1) to the sum of the amounts of a3) and a4) is from 11:1 to 11:8, and
b) spray freeze drying the composition provided in step a) to give the dried antibody composition B.

15. The method of claim 14, wherein the dried antibody composition is in the form of micropellets.

16. The method of claim 14, wherein the salt of arginine is arginine-Cl, arginine-aspartate, arginine-glutamate, arginine-sulfate, arginine-acetate, arginine-succinate, for example wherein the salt of arginine is arginine-Cl.

17. The method of claim 14, wherein the at least one cryoprotectant is selected from the group of cryoprotectants consisting of trehalose, sucrose, sorbitol, glycerol and mannitol, for example wherein the cryoprotectant is trehalose.

18. The method of claim 14, wherein the ratio of the amount of a1) to the sum of the amounts of a3) and a4) is from 11:1 to 11:4, such as from 11:2 to 11:4.

19. The method of claim 14, wherein the aqueous composition A comprises 50 to 150 mg/ml antibody, such as 80 to 120 mg/ml antibody.

20. The method of claim 14, wherein composition A further comprises a5) a buffer, for example a histidine, citrate, acetate, phosphate, Tris, succinate or glycine buffer.

21. The method of claim 14, wherein the antibody is a monoclonal antibody.

22. Dried antibody composition B, obtained or obtainable by the method of claim 14.

23. Use of the dried antibody composition B of claim 14 or an aqueous antibody composition A comprising
a1) an antibody in a concentration which is lower than the concentration of the antibody in an antibody composition I,
a2) at least one surfactant,
a3) at least one cryoprotectant,
a4) at least 0.4% (w/v) arginine, or a salt thereof, wherein the weight (w) is calculated as arginine, such as 0.4 to 3.5% (w/v) arginine
wherein the ratio of the amount of a1) to the sum of the amounts of a3) and a4) is from 11:1 to 11:8
b) drying the composition provided in step a) to yield an antibody composition B, and
c) reconstituting the composition dried in step b)
to give the aqueous antibody composition I
for producing the aqueous antibody composition I comprising at least 200 mg/ml antibody.

24. An aqueous composition A as defined in claim 1.
